(19) United States
(12) Patent Application Publication
(10) Pub. No.: US 2007/0292478 A1
(43) Pub. Date: Dec. 20, 2007

(54) Medical Implant Provided with Inhibitors of Atp Synthesis

(76) Inventor: Popowski Youri, Geneva (CH)

Correspondence Address: KNOBBE MARTENS OLSON & BEAR LLP, 2040 MAIN STREET, FOURTEENTH FLOOR, IRVINE, CA, 92614, US

(21) Appl. No.: 11/662,119

(22) PCT Filed: Aug. 30, 2005
(86) PCT No.: PCT/EP04/09639

§ 371 Date: Feb. 28, 2007

Related U.S. Application Data
(60) Provisional application No. 60/635,778, filed on Dec. 15, 2004.

(30) Foreign Application Priority Data
Dec. 15, 2004 (EP) 04447279.3

(51) Int. Cl. A61F 2/82; A61K 31/06; A61K 31/19; A61K 31/194; A61K 31/352; A61K 38/44; A61K 38/51; A61P 43/00

(52) U.S. Cl. 424/430; 424/436; 424/94.4; 424/94.5; 514/454; 514/557; 514/574; 514/728

(57) ABSTRACT

An implant provided with a composition having at least one type of inhibitor of ATP synthesis is disclosed. The implant is useful for preventing or treating benign or malignant cell proliferation in a duct or a resection cavity of a subject.

### FIELD OF THE INVENTION

The present invention relates to a medical implant disposed with an inhibitor of ATP synthesis for insertion into the cavity of a subject and treating proliferating cells in the wall of the cavity.

### BACKGROUND TO THE INVENTION

Use of implants for the treatment of proliferating cells such as cancer is known in the field of brachytherapy where a radioactive implant is placed in a resectioned cavity after surgical removal of a tumour. The radiation treatment reduces the possibility of tumour regrowth. The implant can be a balloon such as described, for example, in US 2005/0101823, U.S. Pat. No. 5,429,582, where radioactive compositions are introduced to the balloon via a catheter. Alternatively, the implant can be a solid structure coated with a radioactive composition which is placed into a resectioned cavity.

Commonly cytotoxic agents of the art delivered by way of an implant are effective against either residual cancer cells or against established tumours. Cytotoxic agents such as radiation cause shrinkage of proliferating masses, reduce the risk or recurrence in the walls of a resection cavity after a tumorectomy, but damage healthy tissues, induce mutations, which later on may generate radiation induced cancers. This is relevant in areas such as breast cancer. Furthermore, cytotoxic agents may not completely eradicate the tumour—some cells may be located too far from the surface of the implant, and these cells can regrow afterwards.

There is a need for a new method and compositions for treating cellular proliferation in cavities.

### SUMMARY OF SOME EMBODIMENTS OF THE INVENTION

One embodiment of the present invention is a medical implant suitable for insertion into the cavity of a subject provided with a composition comprising at least one type of inhibitor of glycolysis and/or the TCA cycle and/or oxidative phosphorylation, and a slow release agent.

Another embodiment of the present invention is a use of a composition comprising at least one type of inhibitor of glycolysis and/or the TCA cycle and/or oxidative phosphorylation, and a slow release agent, for the preparation of a composition for providing a medical implant for inhibiting cell proliferation in a cavity of a subject.

Another embodiment of the present invention is a use of a composition comprising at least one type of inhibitor of glycolysis and/or the TCA cycle and/or oxidative phosphorylation, and a slow release agent, for the preparation of a composition for providing a medical implant for sensitizing proliferating cells in a cavity of a subject to treatment by radiotherapy.

Another embodiment of the present invention is a use of a composition comprising at least one type of inhibitor of glycolysis and/or the TCA cycle and/or oxidative phosphorylation, and a slow release agent, for the preparation of a composition for providing a medical implant for sensitizing proliferating cells in a cavity of a subject to treatment by chemotherapy.

Another embodiment of the present invention is a kit comprising a) at least one medical implant suitable for insertion into the cavity of a subject and b) a composition comprising at least one type of inhibitor of glycolysis and/or the TCA cycle and/or oxidative phosphorylation, and a slow release agent.

Another embodiment of the present invention is an implant, use or kit as described above, wherein said composition further comprises at least one inhibitor of the PPP.

Another embodiment of the present invention is an implant, use or kit as described above, wherein said implant comprises a material which is bioabsorbable in situ.

Another embodiment of the present invention is an implant, use or kit as described above, wherein said implant comprises a material which is non-bioabsorbable.

Another embodiment of the present invention is an implant, use or kit as described above, wherein implant comprises an inflatable medical balloon.

Another embodiment of the present invention is an implant, use or kit as described above, wherein said balloon is at least partly coated with said composition.

Another embodiment of the present invention is an implant, use or kit as described above, wherein said balloon is at least partly covered with an expandable foil over an expandable region of the balloon, said foil provided with said composition.

Another embodiment of the present invention is an implant, use or kit as described above, wherein said foil is at least partly coated with said composition.

Another embodiment of the present invention is an implant, use or kit as described above, wherein said foil is formed from any of aliphatic polyester copolymers, elastomeric copolymers of epsilon-caprolactone and glycolide, elastomeric copolymers of E-caprolactone and lactide, copolymers of E-caprolactone and lactic acid, elastomeric copolymers of p-dioxanone and lactide, elastomeric copolymers of p-dioxanone and lactic acid, elastomeric copolymers of epsilon-caprolactone and p-dioxanone, elastomeric copolymers of p-dioxanone and trimethylene carbonate, elastomeric copolymers of trimethylene carbonate and glycolide, elastomeric copolymer of trimethylene carbonate and lactide, or elastomeric copolymer of trimethylene carbonate and lactic acid.

Another embodiment of the present invention is an implant, use or kit as described above, wherein said foil comprises a material which is bioabsorbable in situ.

Another embodiment of the present invention is an implant, use or kit as described above, wherein said foil is made from a material which is non-bioabsorbable.

Another embodiment of the present invention is an implant, use or kit as described above, wherein said balloon further comprises a catheter.

Another embodiment of the present invention is an implant, use or kit as described above, wherein said wherein said balloon further comprises one or more catheters are positioned in an geometrical configuration inside the balloon in order to offer optimal dosimetry for a therapy using ionizing or non-ionizing radiation.

Another embodiment of the present invention is an implant, use or kit as described above, wherein said implant is a static implant.

Another embodiment of the present invention is an implant, use or kit as described above, wherein said static implant is at least partly coated with said composition.

Another embodiment of the present invention is an implant, use or kit as described above, wherein said slow release agent is any of magnesium alloys, poly(glycolic) acid, poly(lactic acid) or in general glycolic- and lactic acid based polymers, copolymers, poly caprolactones and in general, poly hydroxyl alkanoate,s poly(hydroxy alcanoic acids), Poly(ethylene glycol), poly vinyl alcohol, poly(orthoesters), poly(anhydrides), poly (carbonates), poly amides, poly imides, poly imines, poly(imino carbonates), poly (ethylene imines), polydioxanes, poly oxyethylene (poly ethylene oxide), poly (phosphazenes), poly sulphones, lipids, poly acrylic acids, poly methylmethacrylate, poly acryl amides, poly acrylo nitriles (Poly cyano acrylates), poly HEMA, poly urethanes, poly olefins, poly styrene, poly terephthalates, poly ethylenes, poly propylenes, poly ether ketones, poly vinylchlorides, poly fluorides, silicones, poly silicates (bioactive glass), siloxanes (Poly dimethyl siloxanes), hydroxyapatites, lactide-capronolactone, natural and non natural poly aminoacids, poly-aminoesters, albumines, alginates, cellulose/cellulose acetates, chitin/chitosan, collagene, fibrine/fibrinogen, gelatine, lignine, proteine based polymers, Poly(lysine), poly(glutamate), poly(malonates), poly (hyaluronic acids), Poly nucleic acids, poly saccharides, poly(hydroxyalkanoates), poly isoprenoids, starch based polymers, copolymers thereof, linear, branched, hyperbranched, dendrimers, crosslinked, functionalised derivatives thereof, hydrogels based on activated polyethyleneglycols combined with alkaline hydrolyzed animal or vegetal proteins.

Another embodiment of the present invention is an implant, use or kit as described above, wherein at least one type of inhibitor of glycolysis and/or the TCA cycle and/or oxidative phosphorylation is encapsulated a micro-capsule or nano-capsule.

Another embodiment of the present invention is an implant, use or kit as described above, wherein a nano-capsule comprises any of a copolymer poly(ethylene oxide) with poly(L-Lactic acid) or with poly(beta-benzyl-L-aspartate); copolymer with poly(lactide-co-glycolide)-[(propylene oxide)-poly(ethylene oxide)]; polyphosphazene derivatives; a poly(ethylene glycol) coated nanosphere; a poly(isobutylcyanoacrylate) nanocapsules poly(gamma-benzyl-L-glutamate)/(poly(ethylene oxide); chitosan-poly(ethylene oxide) nanoparticles; o-carboxymethylate chitosan, or a solid lipid nanosphere.

Another embodiment of the present invention is an implant, use or kit as described above, wherein a micro-capsule comprises any of chitosan; a coated alginate microsphere; an N-(aminoalkyl) chitosan microsphere; a chitosan/calcium alginate bead, a poly(adipic anhydride) microsphere; a gellan-gum bead; a poly(D, L-lactide-co-glycolide) microsphere; an alginate-poly-L-lysine microcapsule; a crosslinked chitosan microsphere; a chitosan/gelatin microsphere; a crosslinked chitosan network bead with spacer groups; an aliphatic polyester; a 1,5-diozepan-2-one microsphere; a D,L-dilactide microsphere; a triglyceride liposphere; a polyelectrolyte complexe of sodium alginate chitosan; a polypeptide microcapsule, or an albumin microsphere.

Another embodiment of the present invention is an implant, use or kit as described above, wherein at least one of said inhibitors is coupled to solubilising agent.

Another embodiment of the present invention is an implant, use or kit as described above, wherein said solubilising agent is cholesterol or derivative thereof.

Another embodiment of the present invention is an implant, use or kit as described above, wherein said cholesterol derivatives are any of cholesteryl-3-betahydroxybutyrate, cholesteryl-halogenated butyrate, cholesteryl-halogenated acetate, cholesteryl-halogenated aceto-acetate, cholesteryl-halogenated acetamide, cholesteryl-halogenated crotonate, cholesteryl-halogenated acetone, cholesteryl-halogenated citrate, or cholesteryl-halogenated oleate

Another embodiment of the present invention is an implant, use or kit as described above, wherein said solubilising agent is vitamin A or derivative thereof.

Another embodiment of the present invention is an implant, use or kit as described above, wherein derivative of vitamin A is formula (IV) or (V): 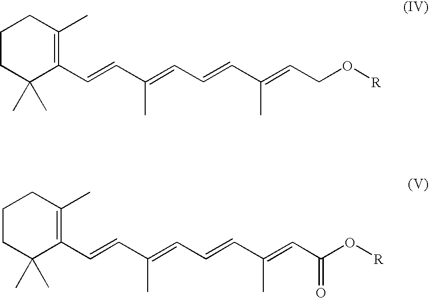
wherein R is selected from the group consisting of betahydroxybutyrate, halogenated butyrate, halogenated acetate, halogenated aceto-acetate, halogenated acetamide, halogenated crotonate, halogenated acetone, halogenated citrate, and halogenated oleate.

Another embodiment of the present invention is an implant, use or kit as described above, wherein said inhibitor of glycolysis inhibits at least one enzyme from the group consisting of hexokinase, glucokinase, phosphoglucose isomerase, phosphofructokinase, aldolase, triose phosphate isomerase, glyceraldehyde 3-phosphate dehydrogenase, phosphoglycerate kinase, phosphoglyceromutase, enolase, pyruvate kinase, and lactate dehydrogenase.

Another embodiment of the present invention is an implant, use or kit as described above, wherein said inhibitor of glycolysis is a hexose sugar modified by removal of the hydroxyl group or by the substitution of the hydroxyl group with halogen atom or thiol at:
C6 for inhibiting hexokinase,
C1 or C2 or C5 for inhibiting phosphoglucoisomerase
C3 and/or C4 for blocking aldolase, and/or
C2 or C3 for blocking glyceraldehyde 3P deshydrogenase, phosphoglycerate kinase, phosphoglycerate mutase, and enolase.

Another embodiment of the present invention is an implant, use or kit as described above, wherein said inhibitor is any of 6-deoxy-6-fluoro-D-glucose, 6-deoxy-6-bromo-D-glucose, 6-deoxy-6-chloro-D-glucose, 6-O-methyl-D-glucose, 6-thio-D-glucose, 6-deoxy-D-glucose, C-6 modified or blocked derivatives of other hexose ring pyranoses, mannopyranoses, galactopyranoses, 6-deoxy-6-fluoro-D-glucose, 6-deoxy-6-bromo-D-mannose, 6-deoxy-6-chloro-D-mannose, 6-deoxy-6-fluoro-D-galactose, 6-deoxy-6-chloro-D-galactose, 6-deoxy-6-iodo-D-galactose, 6-deoxy-6-bromo-D-galactose, halogenated C-6 sugars gluconolactones, glucuronic acid, glucopyranoside, and their phosphate derivatives, glucoronides with halogenated glycosides at the C-1 position, C-2 substituted D-hexoses, 2-deoxy-2-halogeno-D-hexoses, 2-deoxy-2-fluoro-D-glucose, 2-chloro-2-deoxy-D-glucose, 2-bromo-D-glucose, 2-iodo-D-glucose, 2-deoxy-2,2-difluoro-D-arabino-hexose, 2-deoxy-2-fluoro-D-mannose, 2-deoxy-D-arabino-hexose, 2-Deoxy-2-fluoro-D-galactose, 1,6-anhydro-2-deoxy-2-fluoro-beta-D-glucopyranose, 1-6-anhydrosugar, 2-amino-2-deoxy-D-glucose, glucose amine, 2-amino-2-deoxy D-galactose, galactosamine, 2-amino-2-deoxy-D-mannose, mannosamine, 2-deoxy-2-fluoro-D-mannose, 2-deoxy-2-fluoro-D-galactose, 2-deoxy-D-arabino-hexose, 2-deoxy-2,2-difluoro-D-arabino-hexose, 2-deoxy-2-fluoro-D-glucose 1-Phosphate, 2-deoxy-2-fluoro-D-glucose 6-P, 2-deoxy-2-fluoro-D-glucose 1,6 biphosphate, 2-deoxy-2-fluoro-D-mannose 1-P, 2-deoxy-2-fluoro-D-mannose 6-P, 2-deoxy-2-fluoro-D-mannose 1,6-biphosphate, nucleotide diphosphate, uridine di-P, 1-2 deoxy-2-fluoro-D-glucose, C-2-halogen substituted, and NH3 substituted derivatives of D-Glucose 6-phosphate, 2-deoxy-2-fluoro-2-D-glucose-6-phosphate, 2-chloro-2-deoxy-D-glucose-6-phosphate, 2-deoxy-D-arabino-hexose-6-phosphate, D-glucosamine-6-phosphate, 2-deoxy-2-fluoro-2-D-manose-6-P, and any known derivatives, C-2 halogenated derivatives of hexose ring pyranoses, mannopyranoses, galactopyranoses, C-2-deoxy-2-fluoropyranoses, and any derivative, C-2 halogenated sugars derivatives, C-2 fluoro-, bromo-, chloro-, or iodo-sugars derivatives, fluoro, bromo, chloro, or iodo C-2 sugars derivatives, gluconolactones, glucuronic acid, glucopyranoside, and their phosphate derivatives, sugars modified at C-1 or C-5 by replacement of hydroxyl by fluorine or deoxygenation or replacement by a sulfur group, glucosyl fluoride, 1-deoxy-D-glucose, 5-thio-D-glucose, 3-deoxy or 3-fluoro-D-glucose or 4-deoxy or 4-fluoro-D-glucose, 2-fluoro- or 2-iodo-, or 2-thio-, or 2-methoxy- or 3-fluoro-, or 3,3 difluoro-, 3-iodo-, or 3-carboxylo-, or 3-thio-glyceraldehydes or glycerates, 3-fluoro-2-phosphoglycerate, phosphothioesters or other phosphor modified analogs, mannoheptulose mannoheptose, glucoheptose, N-acetylglucosamine, 6-aminonicotinamide acidosis-inducing agents, 2-deoxy-2-fluoro-D-glucose, citrate and halogenated derivatives of citrate, fructose 2,6-bisphosphate, bromoacetylethanolamine phosphate analogues, N-(2-methoxyethyl)-bromoacetamide, N-(2-ethoxyethyl)-bromoacetamide, N-(3-methoxypropyl)-bromoacetamide), iodoacetate, pentalenolactone, arsenic, 1,1-difluoro-3-phosphate-glycerol, oxamate, 2-fluoro-propionic acid or it salts, 2,2-difluoro-propionic acid, pyruvate modified at C-3 such as 3-halo-pyruvate, 3-halopropionic acid, and 2-thiomethylacetic acid, a stereoisomer, tautomer, racemate, prodrug, metabolite thereof, or a pharmaceutically acceptable salt, base, ester or solvate thereof.

Another embodiment of the present invention is an implant, use or kit as described above, wherein said inhibitor of the TCA cycle inhibits at least one enzyme from the group consisting of pyruvate dehydrogenase, citrate synthase, aconitase, isocitrate lyase, alpha-ketoglutarate dehydrogenase complex, succinyl CoA synthetase, succinate dehydrogenase, fumarase, malate synthase, glutaminase, glutamate dehydrogenase, pyruvate dehydrogenase complex and malate dehydrogenase.

Another embodiment of the present invention is an implant, use or kit as described above, wherein said inhibitor of the TCA cycle is any of halo-pyruvate, 3-fluoropyruvate, 3-chloropyruvate, 3-bromopyruvate, 3-iodopyruvate, arsenite, hypoglycin A, methylenecyclopropylacetic acid, alloxan, PNU, p-benzoquinone, fluoroacetate, fluoroacetyl-CoA, halogenated acetyl-CoA, fluoroacetamide, fluorocrotonate, ketone bodies, acetoacetate, hydroxybutyrate, acetone, halogenated ketone bodies, chloroacetoacetate, fluoroacetoacetate, bromoacetoacetate, fluorohydroxybutyrate, chlorohydroxybutyrate, bromohydroxybutyrate, fluoroacetone, chloroacetone, bromoacetone, halogenated acetic acid chloracetic acid, halogenated oleate, fluorocitrate, fluorocitrate 2R, 3R, halogenated citrate, bromocitrate, chlorocitrate, iodocitrate, dichlorovinyl-cysteine, halogenated aminoacids, malonate, pentachlorobutadienyl-cysteine, 2-bromohydroquinone, 3-nitropropionic acid, cis-crotonalide fungicides, 6-diazo-5-oxo-L-norleucine, glu-hydroxyoxamate, p-chloromercuriphenylsulphonic acid, L-glutamate gamma-hydroxamate, p-chloromercuriphenylsulphonic acid, alpha-amino-3-chloro-4,5-dihydro-5-isoxazoleacetic acid, halogenated glutamine, glutamate, a stereoisomer, tautomer, racemate, prodrug, metabolite thereof, or a pharmaceutically acceptable salt, base, ester or solvate thereof
compounds of the general formula:
   X—CH₂—CO—COOH
   wherein X represents a halide, a sulphonate, a carboxylate, an alkoxide, or an amine oxide, or
compounds of the general formula: 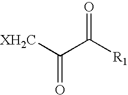
   wherein:
X represents a halide, a sulphonate, a carboxylate, an alkoxide, or an amine oxide,
R₁ may be OR, H, N(R″)2, C1-C6 alkyl, C6-C12 aryl, C1-C6 heteroalkyl, or a C6-C12 heteroaryl.
independently, R″ may represent H, C1-C6 alkyl, or C6-C12 aryl,
independently, R may be H, alkali metal, C1-C6 alkyl, C6-C12 aryl or C(O)R′,
R′ may represent H, C1-C20 alkyl or C6-C12 aryl.

Another embodiment of the present invention is an implant, use or kit as described above, wherein said inhibitor of oxidative phosphorylation is an inhibitor of enzyme complex I.

Another embodiment of the present invention is an implant, use or kit as described above, wherein said inhibitor of complex I is any of tritylthioalanine, caminomycin, piperazinedione, rotenone, amytal, 1-methyl-4-phenylpyridinium, paraquat, methylene blue, or ferricyanide, a stereoisomer, tautomer, racemate, prod rug, metabolite thereof, or a pharmaceutically acceptable salt, base, ester or solvate thereof.

Another embodiment of the present invention is an implant, use or kit as described above, wherein said inhibitor of oxidative phosphorylation is an inhibitor of enzyme complex II.

Another embodiment of the present invention is an implant, use or kit as described above, wherein said inhibitor of oxidative phosphorylation is an inhibitor of complex III

Another embodiment of the present invention is an implant, use or kit as described above, wherein said inhibitor of complex III is any of myxothiazol, antimycin A, ubisemiquinone, cytochrome C, 4,6-diaminotriazine derivatives, metothrexate or electron acceptors such as phenazine methosulfate and 2,6-Dichlorophenol-indophenol, a stereoisomer, tautomer, racemate, prodrug, metabolite thereof, or a pharmaceutically acceptable salt, base, ester or solvate thereof.

Another embodiment of the present invention is an implant, use or kit as described above, wherein said inhibitor of oxidative phosphorylation is an inhibitor of enzyme complex IV.

Another embodiment of the present invention is an implant, use or kit as described above, wherein said inhibitor of enzyme complex IV is any of cyanide, hydrogen sulfide, azide, formate, phosphine, carbon monoxide and electon acceptor ferricyanide a stereoisomer, tautomer, racemate, prodrug, metabolite thereof, or a pharmaceutically acceptable salt, base, ester or solvate thereof.

Another embodiment of the present invention is an implant, use or kit as described above, wherein said inhibitor of oxidative phosphorylation is an inhibitor of enzyme complex V

Another embodiment of the present invention is an implant, use or kit as described above, wherein said inhibitor of enzyme complex V is any of 4′-demethyl-epipodophyllotoxin thenylidene glucoside, tritylthioalanine, caminomycin, piperazinedione, dinitrophenol, dinitrocresol, 2-hydroxy-3-alkyl-1,4-naphtoquinones, apoptolidin aglycone, oligomycin, ossamycin, cytovaricin, naphtoquinone derivatives (e.g. dichloroallyl-lawsone and lapachol), rhodamine, rhodamine 123, rhodamine 6G, arbonyl cyanide p-trifluoromethoxyphenylhydrazone, valinomycin, rothenone, safranine O, cyhexatin, dichlorodiphenyltrichloroethane, chlordecone, arsenate, pentachlorophenol, benzonitrile, thiadiazole herbicides, salicylate, cationic amphilic drugs (amiodarone, perhexyline), gramicidin, calcimycin, pentachlorobutadienyl-cysteine, trifluorocarbonylcyanide phenylhydrazone, a stereoisomer, tautomer, racemate, prodrug, metabolite thereof, or a pharmaceutically acceptable salt, base, ester or solvate thereof.

Another embodiment of the present invention is an implant, use or kit as described above, wherein said inhibitor of oxidative phosphorylation is any of atractyloside, lysophospholipids, n-ethylmaleimide, mersanyl, or p-benzoquinone a stereoisomer, tautomer, racemate, prodrug, metabolite thereof, or a pharmaceutically acceptable salt, base, ester or solvate thereof.

Another embodiment of the present invention is an implant, use or kit as described above, wherein said inhibitor of the PPP inhibits at least one enzyme from the group consisting of glucose-6-phosphate dehydrogenase, lactonase, 6-phosphogluconate dehydrogenase, phosphopentose isomerase, phosphopentose epimerase, transketolase, and transaldolase.

Another embodiment of the present invention is an implant, use or kit as described above, wherein said inhibitor of the PPP is any of 6-aminonicotinamide and 2-Amino-2-deoxy-D-glucose-6-phophate, a stereoisomer, tautomer, racemate, prodrug, metabolite thereof, or a pharmaceutically acceptable salt, base, ester or solvate thereof.

Another embodiment of the present invention is an implant, use or kit as described above, wherein said composition further comprises one or more polymers to facilitate attachment of the composition to the implant and/or facilitate slow release of the composition.

Another embodiment of the present invention is an implant, use or kit as described above, wherein said polymer is one or more of:
aliphatic polyesters, poly(amino acids), copoly(ether-esters), polyalkylenes oxalates, polyamides, poly(iminocarbonates), polyanhydrides, polyorthoesters, polyoxaesters, polyamidoesters, polylactic acid, polyethylene oxide, polycaprolactone, polyhydroxybutyrate valerates, polyoxaesters containing amido groups, poly(anhydrides), polyphosphazenes, silicones, biomolecules and blends thereof,
lactic acid D-,L- and meso lactide, epsilon-caprolactone, glycolide glycolic acid, hydroxybutyrate, hydroxyvalerate, para-dioxanone, trimethylene carbonate and its alkyl derivatives, 1,4-dioxepan-2-one, 1,5-dioxepan-2-one, 6,6-dimethyl-1,4-dioxan-2-one and polymer blends thereof,
polyanhydrides from diacids of the form HOOC—C₆H₄—O—(CH₂)m-O—C₆H₄—COOH wherein m is an integer in the range of from 2 to 8 and copolymers thereof with aliphatic alpha-omega diacids of up to 12 carbons,
naturally enzymatically or are hydrolytically unstable polymers, fibrin, fibrinogen, collagen, gelatin, glycosaminoglycans, elastin, and absorbable biocompatible polysaccharides such as chitosan, starch, fatty acids and esters thereof, glucoso-glycans and hyaluronic acid,
hydrogels
polyurethanes, silicones, poly(meth)acrylates, polyesters, polyalkyl oxides, polyethylene oxide, polyvinyl alcohols, polyethylene glycols and polyvinyl pyrrolidone, as well as, hydrogels, hydrogels from crosslinked polyvinyl pyrrolidinone and polyesters, polyolefins, polyisobutylene and ethylene-alphaolefin copolymers, acrylic polymers, methacrylate and copolymers, vinyl halide polymers and copolymers, polyvinyl chloride, polyvinyl ethers, polyvinyl methyl ether, polyvinylidene polyvinylidene fluoride polyvinylidene chloride, polyacrylonitrile, polyvinyl ketones, polyvinyl aromatics, polystyrene, polyvinyl esters, polyvinyl acetate, copolymers of vinyl monomers with each other and olefins, etheylene-methyl methacrylate copolymers, acrylonitrile-styrene copolymers, ABS resins and ethylene-vinyl acetate copolymers, polyamides, Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose acetate butyrate, cellophane, cellulose nitrate, cellulose propionate, cellulose ethers, carboxymethyl cellulose and hydroxyalkyl celluloses, and combinations thereof,
polyamides of the form-NH—(CH₂)n-CO— and NH—(CH₂)x-NH—CO—(CH₂)y-CO, wherein n is preferably an integer in from 6 to 13; x is an integer in the range of form 6 to 12; and y is an integer in the range of from 4 to 16,
bioabsorbable elastomers, aliphatic polyester elastomers,
elastomeric copolymers of epsilon-caprolactone and glycolide, preferably having a mole ratio of epsilon-caprolactone to glycolide of from about 35:65 to about 65:35, more preferably 45:55 to 35:65,
elastomeric copolymers of epsilon-caprolactone and lactide, L-lactide, D-lactide blends thereof or lactic acid copolymers, preferably having a mole ratio of epsilon-caprolactone to lactide of from about 35:65 to about 90:10 and more preferably from about 35:65 to about 65:35 and most preferably from about 45:55 to 30:70 or from about 90:10 to about 80:20,
elastomeric copolymers of p-dioxanone and lactide including L-lactide, D-lactide and lactic acid, preferably having a mole ratio of p-dioxanone to lactide of from about 40:60 to about 60:40,
elastomeric copolymers of epsilon-caprolactone and p-dioxanone, preferably having a mole ratio of epsilon-caprolactone to p-dioxanone of from about 30:70 to about 70:30,
elastomeric copolymers of p-dioxanone and trimethylene carbonate, preferably having a mole ratio of p-dioxanone to trimethylene carbonate of from about 30:70 to about 70:30,
elastomeric copolymers of trimethylene carbonate and glycolide, preferably having a mole ratio of trimethylene carbonate to glycolide of from about 30:70 to about 70:30,
elastomeric copolymer of trimethylene carbonate and lactide including L-lactide, D-lactide, blends thereof or lactic acid copolymers, preferably having a mole ratio of trimethylene carbonate to lactide of from about 30:70 to about 70:30 and blends thereof.

Another embodiment of the present invention is an implant, use or kit as described above, wherein:
the glycolysis inhibitor is present in a quantity to primarily inhibit the glycolytic pathway, and
the TCA cycle inhibitor, is present in a quantity to secondarily inhibit the TCA cycle.

Another embodiment of the present invention is an implant, use or kit as described above, wherein:
the PPP inhibitor is present in a quantity to primarily inhibit the PPP pathway, and
the TCA cycle or oxidative phosphorylation inhibitor or both, is present in a quantity to secondarily inhibit the TCA cycle and/or oxidative phosphorylation pathways respectively.

Another embodiment of the present invention is an implant, use or kit as described above, suitable for use in inhibiting cell proliferation.

Another embodiment of the present invention is an implant, use or kit as described above, for the preparation of a medical device for inhibiting cell proliferation.

Another embodiment of the present invention is an implant, use or kit as described above, wherein said cell proliferation is cancer.

Another embodiment of the present invention is an implant, use or kit as described above, wherein said cell proliferation is restenosis or stenosis.

Another embodiment of the present invention is an implant, use or kit as described above, wherein the implant is placed in a natural cavity.

Another embodiment of the present invention is an implant, use or kit as described above, wherein said natural cavity is any of artery, vein, bronchial duct, biliary duct, oesophagus, urethral duct, uretheral duct, aeric tract, urogenital tract, nasopharyngeal area, pharynx, small and large bowels, rectum, the trachea, uterine cavity, uterine cervix, vagina, urethra or bladder.

Another embodiment of the present invention is an implant, use or kit as described above, wherein the implant is placed in a resection cavity.

Another embodiment of the present invention is an implant, use or kit as described above, wherein said cavity any of brain tumour resection, breast tumour resection, prostate cancer resection, muscle resection after a sarcoma, uterine laparoscopic myoma resection, head and neck resection cavities, tongue tumour resection, partial upper maxillar resection, liver tumour resection, kidney tumour resection, or bone tumour resection, scar cavity of a melanoma resection or scar of a cheloid resection, or any type of scar.

Another embodiment of the present invention is a method of treating cellular proliferation in a cavity of a subject comprising inserting an implant as defined above into said cavity, said implant configured to contact at least part of the walls of the cavity.

### BRIEF DESCRIPTION OF THE FIGURES

FIG. 1A to K: Longitudinal cross-section drawings of implants of the invention, which comprise an inflatable balloon and one or more catheters. The deflated and corresponding expanded outlines are depicted.

FIGS. 2A and B: Longitudinal cross-section drawings of an implant which is a catheter and balloon covered with an expandable foil. The deflated and corresponding expanded outline is depicted.

FIGS. 3A and I: Cross-sectional drawings of static implants which are solid state structures, in a plurality of forms.

FIG. 4: Cross-sectional drawing of a balloon and catheter in situ, within the oesophagus of a subject.

FIG. 5: Drawing of a balloon and catheter in situ (right) and before placement (left), within the uterus of a subject.

FIG. 6A to F: Drawing of the steps of inserting an implant into a breast resection cavity.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “an inhibitor” means one inhibitor or more than one inhibitor.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of implants, and can also include 1.5, 2, 2.75 and 3.80, when referring to, for example, doses).

The present invention relates to an implant provided with a composition comprising at least one type of inhibitor of ATP synthesis for use in treating proliferating cells present a cavity of a subject.

The present invention also relates to a method for the treatment of proliferating cells present in a cavity of a subject, comprising inserting in the cavity an implant provided with a composition comprising at least one type of inhibitor of ATP synthesis.

The present invention also relates to a method for sensitising proliferating cells present in a cavity of a subject to treatment by radiotherapy and/or chemotherapy, comprising inserting in the cavity an implant provided with a composition comprising at least one type of inhibitor of ATP synthesis prior to said radiotherapy and/or chemotherapy.

The term “proliferating cell” as used herein refers to any type of cell that undergoes unwarranted rapid cell division such as, for example, cancer cells, smooth muscle cells, stenosing cells, restenosing cells, and any rapidly proliferating cell. A collection of such cells form a cell mass.

A cavity can be a natural cavity such as a duct, vascular duct, a bronchial duct, a biliary duct, the oesophasgus, digestive tract, urethral duct, uretheral duct, uterus, stomach, arteries, veins, urethral duct, aeric tract, the urogenital tract, nasopharyngeal area, the pharynx, the small and large bowels, the rectum, the trachea, the uterine cavity, the uterine cervix, the vagina, the urethra, and the bladder or colon. The natural cavity can be any walled cavity of a subject suitable for placing an implant therein. Such cavity may be narrowed by a medical condition such as stenosis, cancer, benign tumours, or invasion of a cancer originating from the wall or passing through the wall of a duct.

The implant may also be placed inside a resection cavity after surgical removal of a malignant or benign proliferating mass. The resection cavities can be any resection cavity in a subject, such as brain tumour resection cavity, liver tumour resection cavity, kidney tumour resection cavity, bone tumour resection cavity, breast tumour resection cavity, prostate cancer resection cavity, muscle resection cavity after a sarcoma resection, uterine laparoscopic myoma resection cavity, head and neck tumour resection cavities (tongue tumour resection, partial upper maxillar resection, etc), scar cavity of a melanoma resection, scar cavity of a cheloid resection or any resection cavity known to someone skilled in the art.

Where an implant is provided with a composition or a composition is used to provide an implant, it means the composition is deposited on, within the implant or on a foil at least partly covering the implant so the composition can be released when the implant contacts the cavity wall. The implant may be coated with the composition, Alternatively, the implant may be impregnated with composition. Alternatively, the implant may comprise cavities in which the composition resides. Alternatively, the implant may be at least partly covered with a foil on or in which the composition is disposed.

The composition is used for the treatment of proliferating cellular masses. The treatment may shrink the mass or may completely eradicate it. The treatment of proliferating cells or tissues may also be applied to regions from which a proliferating mass has been surgically removed to reduce the possibility of relapse or regrowth. The treatment can also be the prevention of regrowth.

The inventors have found that inhibitors of ATP synthesis and the PPP are avidly taken up by proliferating cells. This property enables an inhibitor of cells to be employed proximal to the site of cancerous cells, and to be selectively taken up by said cells in doses higher than by non-cancerous cells. This allows high doses of inhibitors, or extremely potent inhibitors to accumulate inside cancerous cells, while reducing or eliminating cell death of non-proliferating cells. This possibly allows for a reduction in the amount of active substance necessary on/within the implant to inhibit cell proliferation.

The inventors have also found that cell death in cancerous cells can be efficiently effected by administering inhibitors of ATP synthesis and optionally the PPP. The inventors have also found that some inhibitors which block aerobic, anaerobic or both anaerobic and aerobic ATP syntheses are both rapidly and selectively taken up, and are effective at killing rapidly cancerous cells, when part of a cell mass. The inventors have further found that administering inhibitors of ATP synthesis together with some inhibitors of the PPP to proliferating cells leads to a still further effective cell death. An aspect of the invention is an implant that provides at least one inhibitor of ATP synthesis optionally together with at least one inhibitor of the PPP to a mass of proliferating cells. One embodiment of the present invention is a use of one or more inhibitors of ATP synthesis for the preparation of a composition for providing an implant, for the treatment of proliferating cells.

The implant and the controlled release allows treatment of proliferating cells over a prolonged period. It also allows treatment while the patient is fasting, (e.g. every night) and there is no competition towards ATP inhibition from the degradation products of ingested meals.

A “subject” according to the present invention may be any living body susceptible to treatment by an implant. Examples include, but are not limited to humans, dogs, cats, horses, cows, sheep, rabbits, and goats.

A composition as used herein may comprise at least one type of inhibitor of ATP synthesis and optionally at least one inhibitor of the pentose phosphate pathway (PPP). The types of inhibitor and the pathway they inhibit is described in more detail below. In the preferred mode of the invention, an implant is provided with an inhibitor of oxidative phosphorylation which is rhodamine (i.e. rhodamine, rhodamine 6G, rhodamine 123) or dinitrophenol. In another preferred mode of the invention, an implant is provided with an inhibitor of glycolysis which is 2FDG. In another preferred mode of the invention, an implant is provided with an inhibitor of the TCA cycle which is fluoroacetate. In another preferred mode, the implant is provided with a combination of two or more of the aforementioned inhibitors; preferably the implant is provided with rhodamine and 2FDG. In a preferred mode, the implant is used to treat proliferating cells. The therapy may be carried out in combination with low dose radiotherapy and/or chemotherapy.

A composition of the invention may comprise additional substances, such as, for example, those that facilitate the attachment of the inhibitor to the implant, those that release the inhibitor in a controlled manner in situ, and those that facilitate the functioning of the implant in situ. Such additional substances are known to the skilled artisan.

Implant

Implant according to the invention may be any medical implant that can be provided with a composition according to the invention. The surface of the implant, once deployed, is of a shape to at least partly contact the walls of the cavity of the subject. Such implants have been extensively described in the art. The implant may be non-biodegradable. A non-biodegradable implant is generally removed after treatment. The implant may be biodegradable. A biodegradable implant is slowly dissolved while in situ and does not need to be removed after insertion. An implant of the present invention may be temporarily or permanently attached to at least one catheter. The catheter may be used to deliver ionizing or non-ionizing radiation to the implant whose radiation can pass through the wall of the implant. The catheter can be single or multichannel, depending on the requirements of delivery of other therapies. In case several catheters are used, they may be placed in a geometrical disposition in order to warrant optimal geometry for dosimetric purposes.

The composition is present on the surface of the implant, so that once inserted into a subject, the implant releases composition to the surrounding tissues of the cavity. The composition may be coated onto the implant, for example, by spraying, pasting, dipping, electrostatic transfer etc. Alternatively, the composition may be impregnated into the material of the implant structure.

Balloon Implant

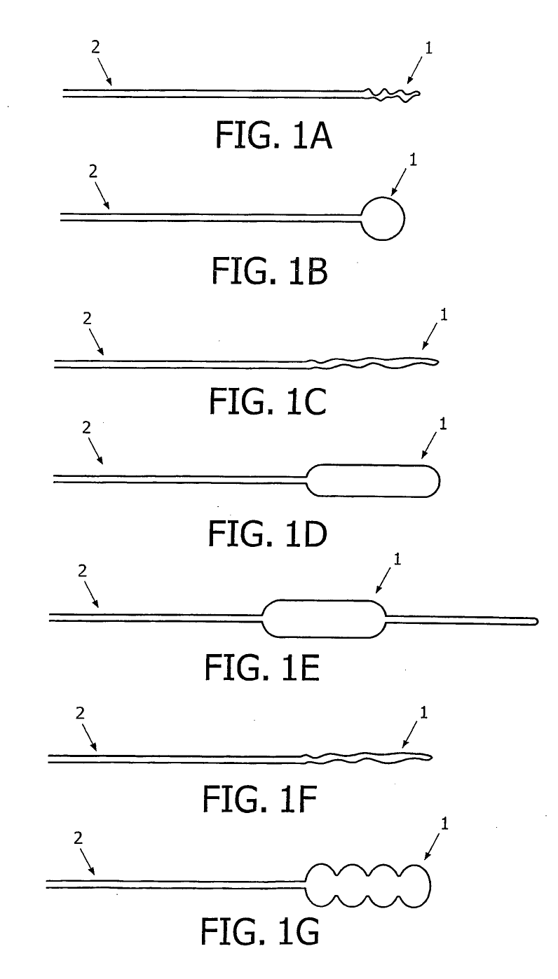

An implant may be an inflatable balloon suitable for insertion into a cavity, which after insertion and inflation at least partly contacts the cavity wall of a subject for the delivery of composition. Various types of balloon are known with a plurality of shapes and features suited, after inflation, to the cavity shape and treatment regime. For example, a balloon after inflation may be longitudinal, ovoid, conical, or it can be made from multiple balloons as indicated in FIG. 1.

With reference to FIG. 1, a balloon 1 of the present invention may be temporarily or permanently attached to at least one catheter 2. The catheter 1 allows inflation of the balloon after placement in the cavity. Once in the cavity and after inflation, the catheter 2 can be removed, or it can remain in place, to provide, for example, additional treatments through the catheter tubing. For example, the catheter may also be used to deliver heating, cooling or ionizing or non-ionizing radiation to the balloon whose radiation can pass through the wall of the balloon. The catheter can be single or multichannel, depending on the requirements of inflation and delivery of other substances. FIG. 1K depicts a spherical balloon connected to two catheters which may be used to inflate and circulate liquid or gaseous medium to the interior of the balloon. In case several catheters are used, they may be placed in a geometrical disposition in order to warrant optimal geometry. The catheter 2 may provide additional substances to the subject, for example, where the balloon 1 is inserted into the oesophagus of a subject, liquid nourishment may be fed into the stomach via the catheter 2. In the deflated state (FIG. 1 A, C, F, H), the collapsed balloon 1 can be readily inserted into the cavity. It also may be guided along blood vessels, or ducts such as bronchial ducts to reach the desired location. Once in situ, the balloon 1 may be inflated by providing an inflation medium to the catheter 2, such as air, saline solution or contrast medium. The balloon may be configured to adopt a shape in the inflated state suited to the shape of the cavity. In this regard FIG. 1 shows a spherical balloon 1 (B, K), cylindrical balloon 1 (D, E), multi-ribbed balloon 1 (G) and a conical balloon 1 (I, J), each attached to a catheter 2. Other suitable balloon shape or arrangement of catheter can be employed, depending on the particular application.

A balloon is made of biocompatible materials which may include biostable and/or bioabsorbable materials. Suitable biocompatible biostable materials include, but are not limited to polyamide 11 or 12. The balloon may be inserted for the duration of treatment and later removed. The balloon can be constructed of materials sufficiently flexible so as to be able to follow and conform to the shape of the cavity, such as latex rubber, elastic, or plastic.

Foil

According to one embodiment of the invention, the balloon is at least covered with an expandable foil on or in which the composition is disposed. The foil allows the composition to be readily applied to any available balloon, without the need to apply a liquid coating of the composition, which can be laborious and take time to prepare. The foil can be supplied as a sheath for covering the uninflated balloon. Alternatively, it can be supplied as a sheet that can be cut to size and attached to the balloon. The foil may be formed from a bioabsorbable or non-bioabsorbable material.

The foil may be essentially the same size and shape as the uninflated balloon, or slightly larger. For example, a 5 cm long conical deflated balloon may be covered with a foil 7 or 8 cm long. Once inflated and conical, the foil would cover the conical balloon adequately. This balloon may be used to treat intra-uterine lesions for instance.

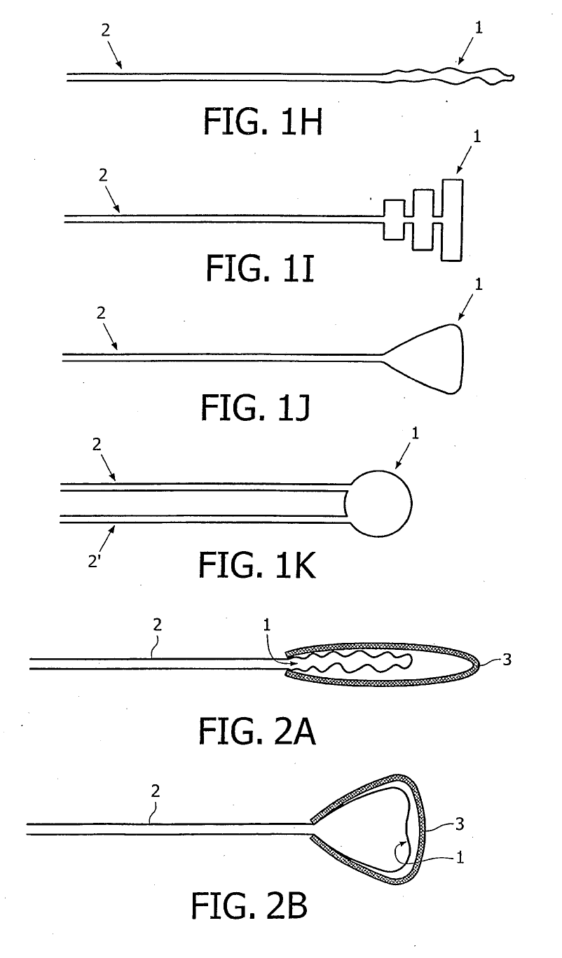

With reference to FIG. 2, the unexpanded foil 3, may be a sheath shaped to closely follow the shape of the uninflated balloon 1, or may be slightly larger. After inflation of the balloon 1 by filling with, for example, saline solution through the catheter 2, the foil 3 is expanded by force of the expanding balloon.

The foil can be made from any suitable expandable material. Examples of suitable expandable materials include aliphatic polyester elastomers. In the proper proportions aliphatic polyester copolymers are expandable. Examples of suitable bioabsorbable expandable polymers are described in U.S. Pat. No. 5,468,253 hereby incorporated by reference. Preferably the bioabsorbable biocompatible expandable polymers are based on aliphatic polyester, including but not limited to those selected from the group consisting of elastomeric copolymers of epsilon-caprolactone and glycolide (preferably having a mole ratio of epsilon-caprolactone to glycolide of from about 35:65 to about 65:35, more preferably 45:55 to 35:65) elastomeric copolymers of E-caprolactone and lactide, including L-lactide; D-lactide blends thereof or lactic acid copolymers (preferably having a mole ratio of epsilon-caprolactone to lactide of from about 35:65 to about 90:10 and more preferably from about 35:65 to about 65:35 and most preferably from about 45:55 to 30:70 or from about 90:10 to about 80:20) elastomeric copolymers of p-dioxanone (1,4-dioxan-2-one) and lactide including L-lactide, D-lactide and lactic acid (preferably having a mole ratio of p-dioxanone to lactide of from about 30:70 to about 70:30, 45:55 to about 55:45, and preferably from about 40:60 to about 60:40) elastomeric copolymers of epsilon-caprolactone and p-dioxanone (preferably having a mole ratio of epsilon-caprolactone to p-dioxanone of from about 40:60 to about 60:40 and preferably from about 30:70 to about 70:30) elastomeric copolymers of p-dioxanone and trimethylene carbonate (preferably having a mole ratio of p-dioxanone to trimethylene carbonate of from about 40:60 to about 60:40, and preferably from about 30:70 to about 70:30), elastomeric copolymers of trimethylene carbonate and glycolide (preferably having a mole ratio of trimethylene carbonate to glycolide of from about 40:60 to about 60:40 and preferably from about 30:70 to about 70:30), elastomeric copolymer of trimethylene carbonate and lactide including L-lactide, D-lactide, blends thereof or lactic acid copolymers (preferably having a mole ratio of trimethylene carbonate to lactide of from about 30:70 to about 70:30) and blends thereof. As is well known in the art these aliphatic polyester copolymers have different hydrolysis rates, therefore, the choice of expandable polymers may in part be based on the requirements for the foil. For example epsilon-caprolactone-co-glycolide copolymer (45:55 mole percent, respectively) films lose 90% of their initial strength after 2 weeks in simulated physiological buffer whereas the epsilon-caprolactone-co-lactide copolymers (40:60 mole percent, respectively) loses all of its strength between 12 and 16 weeks in the same buffer. Preferably the foil may be made from hydrogels based on activated polyethyleneglycols (PEGs) combined with alkaline hydrolyzed animal or vegetal proteins.

Mixtures of the fast hydrolyzing and slow hydrolyzing polymers can be used to adjust the time of strength retention.

Examples of balloons include, but are not limited to, those described in US 2005/101823, WO 92/22350, U.S. Pat. No. 5,429,582, U.S. Pat. No. 4,763,642, U.S. Pat. No. 6,673,006, U.S. Pat. No. 6,589,158, U.S. Pat. No. 6,537,194, U.S. Pat. No. 6,482,142, U.S. Pat. No. 6,413,204, U.S. Pat. No. 6,238,374, U.S. Pat. No. 6,200,257, U.S. Pat. No. 6,083,148, U.S. Pat. No. 6,022,308, U.S. Pat. No. 5,931,774, U.S. Pat. No. 5,913,813. The content of all documents referred to in this application are incorporated herein by reference.

Static Implant

A static implant is a flexible or non-flexible biocompatible structure suitable for insertion into a cavity of a subject, the surface of the implant after insertion at least partly contacting the cavity wall for the delivery of composition. Unlike a balloon, a static implant does not have means for manual expansion. Implants comprising materials that naturally swell, for example in a moist environment are also considered static implants. Bioabsorable implants which gradually dissolve over time are also static implants. Various types of static implants are known with a plurality of shapes and features suited to the cavity shape and treatment regime. For example, a static implant can be hollow, solid, have a continuous surface, be pitted with holes, comprise at least two joined elements or a combination of these features. A static implant may be shaped to suit the cavity so that the surface of the implant at least partly contacts the cavity wall. It may be pre-shaped, or can be shaped by the practitioner for example by cutting or bending.

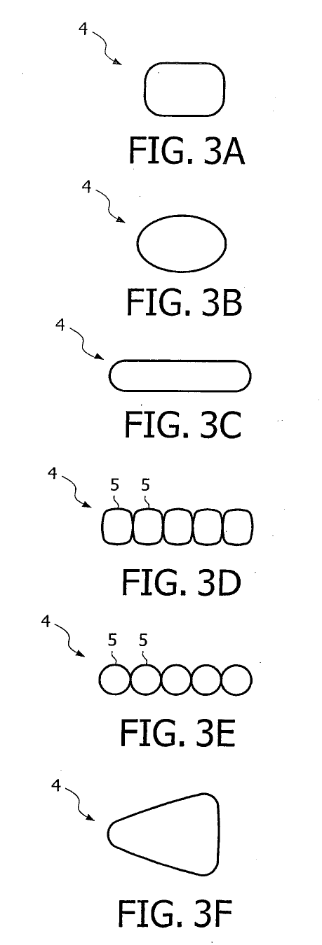

With reference to FIG. 3, a static implant 4 may be a solid or hollow cylinder (A), oval (B), elongate cylinder (C), or cone (F). A static implant 4 may comprise at least two joined elements such as spheres (E) or rounded cubes (D). A static implant 4 may be provided with a plurality of openings such as shown in FIG. 1G where the implant is a hollow cylinder. A static implant 4 may be crossed with at least one catheter 2 as shown in FIG. 3H. A static implant 4 may be provided with one or more handles 7 as depicted in FIG. 3I, where the implant is a solid or hollow oval.

A static implant is made of biocompatible materials which may include biostable and/or bioabsorbable materials. The material of the static implant is amendable to coating with composition, or being impregnated therewith. Suitable biocompatible metals include, but are not limited to, stainless steel, tantalum, titanium alloys (including nitinol), and cobalt alloys (including cobalt-chromium-nickel alloys). A static implant may be made of biocompatible and bioabsorbable materials such as magnesium based alloys.

Suitable nonmetallic biocompatible materials include, but are not limited to, polyamides, polyolefins (i.e. polypropylene, polyethylene etc.), nonabsorbable polyesters (i.e. polyethylene terephthalate), and bioabsorbable aliphatic polyesters (i.e. homopolymers and copolymers of lactic acid, glycolic acid, lactide, glycolide, para-dioxanone, trimethylene carbonate, epsilon-caprolactone, lactide capronolactone etc. and blends thereof), poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phosphate ester), poly(amino acid), poly(hydroxy butyrate), polyacrylate, polyacrylamid, poly(hydroxyethyl methacrylate), elastin polypeptide co-polymer, polyurethane, starch, polysiloxane and their copolymers.

Bioabsorbable static implants may be inserted at the site of treatment, and left in place. The static implant does not become incorporated into the wall of the cavity being treated, but is dissolved during and/or after treatment. For instance, an implant is made from poly(D, L-lactide-co-glycolide) (PLA/PGA), a 2.5 cm diameter cylinder, 44 mm long, is coated with a 100 micron foil of lactide-capronolactone containing the active substances. This cylinder may be sutured inside the cavity left by the resection of a breast tumour. Once the lactide foil has degraded in 4 to 6 weeks and has liberated its active substances, the PLA/PGA device starts degrading and disappears in the 3-4 following months. The use of a device sutured to the resection cavity walls allows to be sure that optimal contact exists between the template and the drug delivery system. Once the drug has been delivered, the support system may disappear.

Where the static implant is made from biostable (non-absorbable) materials, it may be inserted for the duration of treatment and later removed.

Polymers

It is an aspect of the invention that the implant is provided with at least one type of inhibitor of ATP synthesis and optionally at least one inhibitor of the PPP by way of at least partially coating the implant with a composition comprising a polymer. A polymer according to the present invention is any that facilitates attachment of the inhibitor(s) to the implant (i.e. implant and/or foil), and/or facilitates the controlled release of said inhibitors.

Polymers suitable for use in the present invention are any that are capable of attaching to or being impregnated within the implant and releasing inhibitor. They must be biocompatible to minimize irritation to the cavity wall. Polymers may be, for example, film-forming polymers that are absorbable or non-absorbable. The polymer may be biostable or bioabsorbable depending on the desired rate of release or the desired degree of polymer stability. Preferably, the polymer bioabsorbs simultaneously with the implant itself. Preferably, the polymer may bioabsorb first so releasing inhibitor, followed by the implant.

Suitable bioabsorbable polymers that could be used include polymers selected from the group consisting of aliphatic polyesters, poly(amino acids), copoly(ether-esters), polyalkylenes oxalates, polyamides, poly(iminocarbonates), polyanhydrides, polyorthoesters, polyoxaesters, polyamidoesters, polylactic acid (PLA), polyethylene oxide (PEO), polycaprolactone (PCL), polyhydroxybutyrate valerates, polyoxaesters containing amido groups, poly(anhydrides), polyphosphazenes, silicones, hydrogels, biomolecules and blends thereof.

For the purpose of the present invention, aliphatic polyesters include homopolymers and copolymers of lactide (which includes lactic acid D-, L- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and polymer blends thereof. Poly(iminocarbonate) for the purpose of this invention include as described by Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in Journal of Biomaterials Research, Vol. 22, pages 993-1009, 1988 by Cohn and Younes and Cohn, Polymer Preprints (ACS Division of Polymer Chemistry) Vol. 30(1), page 498, 1989 (e.g. PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein).

Polyphosphazenes, co-, ter- and higher order mixed monomer based polymers made from L-lactide, D,L-lactide, lactic acid, glycolide, glycolic acid, para-dioxanone, trimethylene carbonate and epsilon-caprolactone such as are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein).

Polyanhydrides from diacids of the form HOOC—C₆H₄—O—(CH₂)ₘ—O—C₆H₄—COOH wherein m is an integer in the range of from 1 to 11, 3 to 9, 3 to 7, 2 to 6 or preferably 2 to 8, and copolymers thereof with aliphatic alpha-omega diacids of up to 8, 9, 10, 11 or preferably 12 carbons. Polyoxaesters polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference).

Other polymeric biomolecules for the purpose of this invention include naturally occurring materials that may be enzymatically degraded in the human body or are hydrolytically unstable in the human body such as fibrin, fibrinogen, collagen, gelatin, glycosaminoglycans, elastin, and absorbable biocompatible polysaccharides such as chitosan, starch, fatty acids (and esters thereof), glucoso-glycans and hyaluronic acid.

Suitable biostable polymers with relatively low chronic tissue response, such as polyurethanes, silicones, poly(meth)acrylates, polyesters, polyalkyl oxides (polyethylene oxide), polyvinyl alcohols, polyethylene glycols and polyvinyl pyrrolidone, as well as, hydrogels such as those formed from crosslinked polyvinyl pyrrolidinone and polyesters could also be used. Other polymers could also be used if they can be dissolved, cured or polymerized on the implant. These include polyolefins, polyisobutylene and ethylene-alphaolefin copolymers; acrylic polymers (including methacrylate) and copolymers, vinyl halide polymers and copolymers, such as polyvinyl chloride; polyvinyl ethers, such as polyvinyl methyl ether; polyvinylidene halides such as, polyvinylidene fluoride and polyvinylidene chloride; polyacrylonitrile, polyvinyl ketones; polyvinyl aromatics such as polystyrene; polyvinyl esters such as polyvinyl acetate; copolymers of vinyl monomers with each other and olefins, such as etheylene-methyl methacrylate copolymers, acrylonitrile-styrene copolymers, ABS resins and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e. carboxymethyl cellulose and hydroxyalkyl celluloses); and combinations thereof. Polyamides for the purpose of this application would also include polyamides of the form-NH—(CH₂)n-CO— and NH—(CH₂)x-NH—CO—(CH₂)y-CO, wherein

n is an integer in from 5 to 15, 7 to 11, 8 to 10 or preferably 6 to 13;

x is an integer in the range of from 5 to 14, 7 to 11, 8 to 10 or preferably 6 to 12; and

y is an integer in the range of from 3 to 18, 5 to 14, 6 to 10 or preferably 4 to 16. The list provided above is illustrative but not limiting.

Other polymers may be made from hydrogels based on activated polyethyleneglycols (PEGs) combined with alkaline hydrolyzed animal or vegetal proteins.

Other polymers suitable for use in the present invention are bioabsorbable elastomers, more preferably aliphatic polyester elastomers. In the proper proportions aliphatic polyester copolymers are elastomers. Elastomers present the advantage that they tend to adhere well to the implant and can withstand significant deformation without cracking. The high elongation and good adhesion provide superior performance to other polymer coatings when the balloon is expanded. Examples of suitable bioabsorbable elastomers are described in U.S. Pat. No. 5,468,253 hereby incorporated by reference. Preferably the bioabsorbable biocompatible elastomers based on aliphatic polyester, including but not limited to those selected from the group consisting of elastomeric copolymers of epsilon-caprolactone and glycolide (preferably having a mole ratio of epsilon-caprolactone to glycolide of from about 35:65 to about 65:35, more preferably 45:55 to 35:65) elastomeric copolymers of E-caprolactone and lactide, including L-lactide, D-lactide blends thereof or lactic acid copolymers (preferably having a mole ratio of epsilon-caprolactone to lactide of from about 35:65 to about 90:10 and more preferably from about 35:65 to about 65:35 and most preferably from about 45:55 to 30:70 or from about 90:10 to about 80:20) elastomeric copolymers of p-dioxanone (1,4-dioxan-2-one) and lactide including L-lactide, D-lactide and lactic acid (preferably having a mole ratio of p-dioxanone to lactide of from about 30:70 to about 70:30, 45:55 to about 55:45, and preferably from about 40:60 to about 60:40) elastomeric copolymers of epsilon-caprolactone and p-dioxanone (preferably having a mole ratio of epsilon-caprolactone to p-dioxanone of from about 40:60 to about 60:40 and preferably from about 30:70 to about 70:30) elastomeric copolymers of p-dioxanone and trimethylene carbonate (preferably having a mole ratio of p-dioxanone to trimethylene carbonate of from about 40:60 to about 60:40, and preferably from about 30:70 to about 70:30), elastomeric copolymers of trimethylene carbonate and glycolide (preferably having a mole ratio of trimethylene carbonate to glycolide of from about 40:60 to about 60:40 and preferably from about 30:70 to about 70:30), elastomeric copolymer of trimethylene carbonate and lactide including L-lactide, D-lactide, blends thereof or lactic acid copolymers (preferably having a mole ratio of trimethylene carbonate to lactide of from about 30:70 to about 70:30) and blends thereof. As is well known in the art these aliphatic polyester copolymers have different hydrolysis rates, therefore, the choice of elastomer may in part be based on the requirements for the coatings adsorption. For example epsilon-caprolactone-co-glycolide copolymer (45:55 mole percent, respectively) films lose 90% of their initial strength after 2 weeks in simulated physiological buffer whereas the epsilon-caprolactone-co-lactide copolymers (40:60 mole percent, respectively) loses all of its strength between 12 and 16 weeks in the same buffer. Mixtures of the fast hydrolyzing and slow hydrolyzing polymers can be used to adjust the time of strength retention.

The amount of coating may range from about 0.5 to about 20 as a percent of the total weight of the implant after coating and preferably will range from about 1 to about 15 percent. The polymer coatings may be applied in one or more coating steps depending on the amount of polymer to be applied. Different polymers may also be used for different layers in the implant coating. In fact it may be an option to use a dilute first coating solution as primer to promote adhesion of a subsequent coating layers that may contain inhibitor.

Additionally, a top coating can be applied to further delay release of the inhibitor, or they could be used as the matrix for the delivery of a different pharmaceutically active material. The amount of top coatings on the implant may vary depending on the volume of the implant, but will generally be less than about 1 gram per square cm of implant surface, preferably the amount of top coating will be in the range of about micrograms to about 500 micrograms per square cm and most preferably in the range of from about 50 micrograms to 500 about micrograms per square cm of implant surface. Layering of coating of fast and slow hydrolyzing copolymers can be used to stage release of the drug or to control release of different agents placed in different layers. Polymer blends may also be used to control the release rate of different agents or to provide desirable balance of coating (i.e. elasticity, toughness etc.) and drug delivery characteristics (release profile). Polymers with different solubilities in solvents can be used to build up different polymer layers that may be used to deliver different drugs or control the release profile of a drug. For example since epsilon-caprolactone-co-lactide elastomers are soluble in ethyl acetate and epsilon-caprolactone-co-glycolide elastomers are not soluble in ethyl acetate. A first layer of epsilon-caprolactone-co-glycolide elastomer containing a drug can be over coated with epsilon-caprolactone-co-glycolide elastomer using a coating solution made with ethyl acetate as the solvent. Additionally, different monomer ratios within a copolymer, polymer structure or molecular weights may result in different solubilities. For example, 45/55 epsilon-caprolactone-co-glycolide at room temperature is soluble in acetone whereas a similar molecular weight copolymer of 35/65 epsilon-caprolactone-co-glycolide is substantially insoluble within a 4 weight percent solution. The second coating (or multiple additional coatings) can be used as a top coating to delay the drug delivery of the drug contained in the first layer. Alternatively, the second layer could contain a different inhibitor to provide for sequential inhibitor delivery. Multiple layers of different inhibitors could be provided by alternating layers of first one polymer then the other. As will be readily appreciated by those skilled in the art numerous layering approaches can be used to provide the desired drug delivery.

The coatings can be applied by suitable methodology known to the skilled person, such as, for example, dip coating, spray coating, electrostatic coating, melting a powered form onto the implant. The coating may also be applied during the intervention by the interventional cardiologist on a bare implant. As some polymers (for instance polyorthoesters) need special conservation conditions (argon atmosphere and cold temperature), the drug with the coating may be delivered in a special packing. The MD would apply the coating on the bare implant surface—as it is slightly sticky—just before introducing the implant inside the cavity.

Other examples of polymeric coatings, and coating methods are given in patent documents EP 1 107 707, WO 97/10011, U.S. Pat. No. 6,656,156, EP 0 822 788, U.S. Pat. No. 6,364,903, U.S. Pat. No. 6,231,600, U.S. Pat. No. 5,837,313, WO 96/32907, EP 0 832,655, U.S. Pat. No. 6,653,426, U.S. Pat. No. 6,569,195, EP 0 822 788 B1, WO 00/32238, U.S. Pat. No. 6,258,121, EP 0 832,665, WO 01/37892, U.S. Pat. No. 6,585,764, U.S. Pat. No. 6,153,252 which are incorporated herein by reference.

Non-Polymeric Coatings

Another aspect of the invention is an implant provided with a composition of the invention, wherein the presence of a polymer is optional. Such implant suited to polymeric and non-polymeric coatings and compositions are known in the art. These implants may, for example, have a rough surface, microscopic pits or be constructed from a porous material.

Biodegradable Implants

As already mentioned above, an implant may be biodegradable and provided with a composition according to the present invention. The composition may be coated onto the implant or impregnated into the implant structure, said composition released in situ concomitant with the biodegradation of the implant. Suitable materials for the main body of a static implant includes, but are not limited to poly(alpha-hydroxy acid) such as poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen or other connective proteins or natural materials, poly(hydroxybutyrate), polyanhydride, polyphosphoester, poly(amino acids), hylauric acid, starch, chitosan, adhesive proteins, co-polymers of these materials as well as composites and combinations thereof and combinations of other biodegradable polymers. Biodegradable glass or bioactive glass is also a suitable biodegradable material for use in the present invention. A composition of the present invention may be incorporated into a biodegradable implant using known methods. Biodegradable implants may also be made from a metal (lanthanide such as, but not limited to magnesium, or magnesium alloy), or an association of organic and non-organic material (such as, but not limited to a magnesium based alloy combined with a polymer).

Medical Treatments

The inventors have found that an advantage of using compounds that inhibit ATP synthesis and compounds that inhibit the PPP when present, is that they are selectively absorbed by the rapidly proliferating cells.

According to the present invention, an implant may be placed on or adjacent to tumoral tissues for treatment of a tumour. Examples of tumours suitable for treatment according to the invention include biliary tract adenocarcinoma, esophageal epidermoid or adenocarcinoma, colon and rectum adenocarcinoma, bronchial epidermoid or adeno-carcinomas, uterine, prostate, brain, breast, muscle resection, head and neck (tongue, maxillar) cancers, melanomas etc. Examples of precancerous lesions include and are not limited to Barett esophageal displasia, vaginal displasia, etc. Rapidly dividing precancerous or tumour cells thus take up the inhibitory compounds according to the invention, so leading to cell death. An implant of the invention may be introduced at an early stage, for example, in the treatment of esophageal cancer, where only superficial lesions are present. The implant can be further removed and the lesion be followed-up by esophagoscopy.

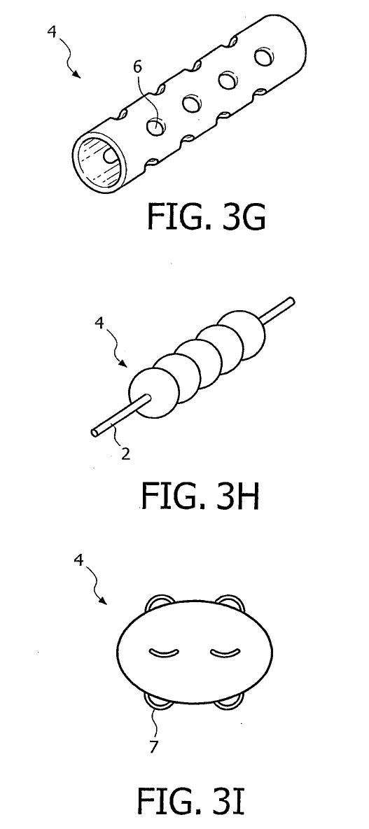

With reference to FIG. 4, an implant which is an inflatable balloon 1 may be inserted into a cavity which as the oesophagus 10 of a subject in the vicinity of a tumour 13, 13′. After inflation, the balloon contacts the oesophageal wall, delivering composition to the cancerous cells. The catheter in this instance also finishes in a feeding tube 8, allowing nourishment or other medicaments to be provided directly to the stomach 9 of the subject during treatment. The treatment may be combined with other cytotoxic therapies such as chemotherapy or radiotherapy.

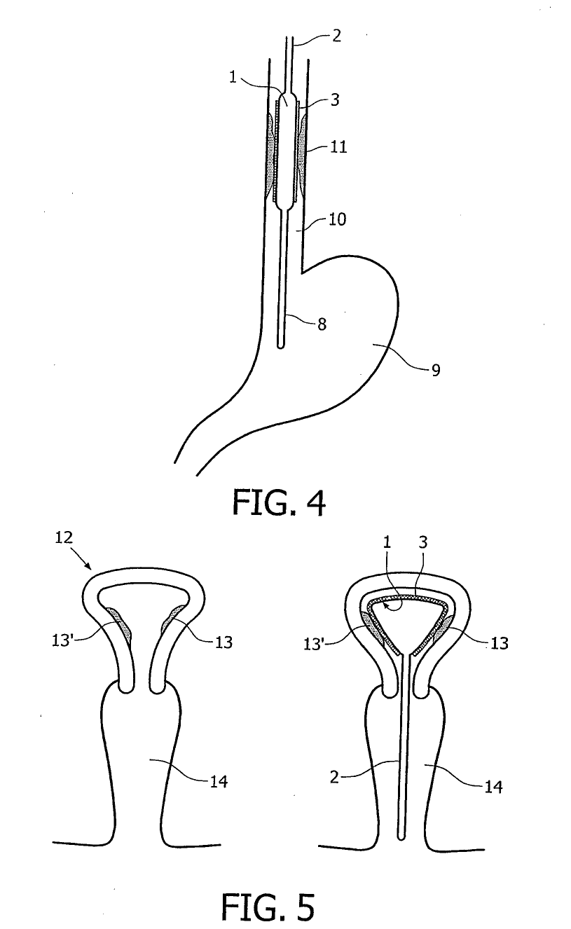

A similar example is depicted in FIG. 5 where a cancer 13 is present on the wall of the uterus 12 (left figure). A balloon 1 is inserted through the vagina 14 (right figure) and through the cervical canal, and inflated. The balloon 1 is provided with a foil 3 onto which the composition is disposed. The shape of the balloon 1 essentially matches the shape of the uterine cavity so the foil 3 contacts the wall of the uterus without undue pressure on the uterus.

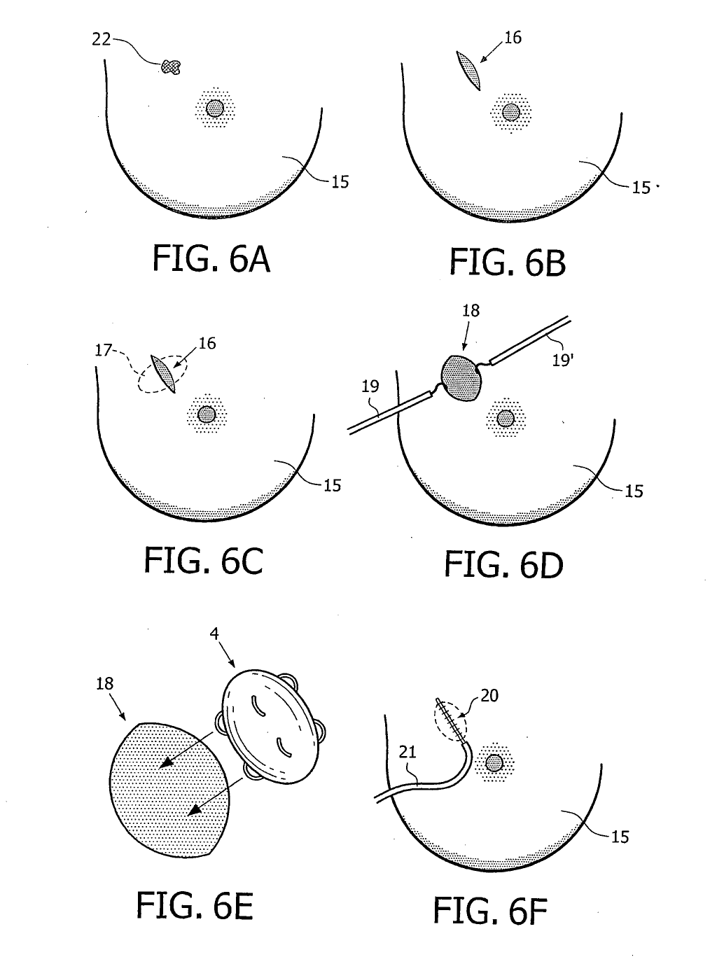

FIG. 6 shows steps in a treatment of cancer whereby an implant is inserted into a resectioned cavity after excision of a breast tumour. The cancer 22 is identified (A) in the breast 15 of a subject. An incision 16 is made in the breast 15 (B) and the growth removed. A cavity 17 remains (C). The wound 18 is opened by clamps 19 and 19′ in step D, and a static implant 4 with handles inserted therein (E). The wound is closed by stitched in step F. Optionally, the implant 4 may be crossed by one to several catheters 21, for example, where the implant is an inflatable balloon. Said catheter 21 may provide other medicaments or heat or ionizing radiation.

The implant may also be placed in situ after the removal of a tumour. For example, after surgical removal of an oesophageal cancer, an implant may be placed in the area of the suture to kill cells possibly remaining after surgery. Similarly, an implant may be placed in a resectioned cavity after removal of a tumour e.g. after removal of a brain cancer or a prostrate cancer

The present invention is useful for treating any animal in need including humans, livestock, domestic animals, wild animals, or any animal in need of treatment. Examples of an animal is human, horse, cat, dog, mice, rat, gerbil, bovine species, pig, fowl, camelidae species, goat, sheep, rabbit, hare, bird, elephant, monkey, chimpanzee etc. An animal may be a mammal.

ATP Synthesis Inhibitors

The ATP synthesis inhibitors of the present of the invention may be any inhibitor that inhibits a pathway directly or indirectly leading to ATP synthesis, or derivatives thereof, or salts thereof. The composition of the present invention comprise an inhibitor of glycolysis or TCA cycle, or oxidative phosphorylation. The composition may comprise inhibitors towards any two or more of the above mentioned pathways. The composition may optionally comprise an inhibitor of the PPP.

Glycolysis

An example of a pathway involved in anaerobic ATP synthesis is glycolysis. Enzymes associated with this pathway are known in the art and include hexokinase, glucokinase (in tumors or in rapidly proliferating tissues), phosphoglucose isomerase, phosphofructokinase, aldolase, triose phosphate isomerase, glyceraldehydes 3-phosphate dehydrogenase, phosphoglycerate kinase, phosphoglyceromutase, enolase, pyruvate kinase and, indirectly, lactate dehydrogenase (lactate metabolism). It is an aspect of the invention that an inhibitor of anaerobic ATP synthesis is an inhibitor of an enzyme associated with the glycolytic pathway. Inhibitors of the glycolytic pathway are any known in the art.

Inhibitors of hexokinase may be configurational isomers of monosaccharides modified at C-6 by substitution (replacement) or removal of 6-OH (the hydroxyl group). An example of a substituent is a blocking moiety—for example, an atom from the halogen family, such as fluorine (6-fluoro-D-glucose). Another example of a substituent is a thiol group. Monosaccharides modified at C-6 by removal of 6-OH will not be transformed by hexokinase or glucokinase (see below) to glucose-6-phosphate and can potentially block both enzymes.

Inhibitors of hexokinase are any known in the art and may include, but are not limited to any of the following:
6-fluoro-D-glucose, 6-bromo-D-glucose, 6-chloro-D-glucose, 6-O-methyl-D-glucose, 6-Thio-D-glucose, 6-deoxy-D-glucose, and any derivative known in the art.
C-6 substituted derivatives of other hexose ring pyranoses (mannopyranoses, galactopyranoses). Examples include 6-deoxy-6-fluoro-D-mannose, and any known in the art.
Various halogenated (fluoro, bromo, chloro-) C6 sugars derivatives such as gluconolactones, glucuronic acid, glucopyranoside, and their phosphate derivatives, and any known in the art. Halogenated glucosides may also be delivered indirectly to the cell, by compounds such as glucoronides with halogenated glycosides at the C1 position (once in the cell, glucoronidases will cleave it, and deliver active hexose in the cell). Preferably, an inhibitor of hexokinase is 6-deoxy-6-fluoro-D-glucose and its derivatives.

Inhibitors of glucokinase may be any in the art. They include, and are not limited to mannoheptulose, mannoheptose, glucoheptose, N-acetylglucosamine. Glucokinase is predominantly present in tumours only.

Inhibitors of phosphoglucoisomerase: Phosphoglucoseisomerase transforms glucose 6-phosphate to fructose 6-phosphate. Such transformation requires the presence of an hydroxyl group at C-2. Therefore, analogs without hydroxyl or having the hydroxyl properly blocked will not undergo isomerization by phosphoglucose isomerase.

Another way to inhibit isomerization by phosphoglucose isomerase is by modifying the glucose 6-phosphate at C-1 or C-5 by substituting hydroxyl with a halogenated atom (fluorine, glucosyl fluoride), or by simple deoxygenation to 1-deoxy-D-glucose.

Inhibitors of phosphoglucoisomerase are any known in the art and may include, but are not limited to any of the following:

C2 substituted D-hexoses, such as 2-deoxy-2-halogeno-D-hexoses, such as 2-deoxy-2-fluoro-D-glucose (2FDG), 2-chloro-2-deoxy-D-glucose, 2-bromo-D-glucose, 2-iodo-D-glucose, 2-deoxy-2,2-difluoro-D-arabino-hexose, 2-deoxy-2-fluoro-D-mannose, 2-deoxy-D-arabino-hexose, 2-Deoxy-2-fluoro-D-galactose, 1,6-anhydro-2-deoxy-2-fluoro-beta-D-glucopyranose (1-6-anhydrosugar), 2-amino-2-deoxy-D-glucose (glucose amine), 2-amino-2-deoxy D galactose (galactosamine), 2-amino-2-deoxy-D-mannose (mannosamine), 2-deoxy-2-fluoro-D-mannose, 2-deoxy-2-fluoro-D-galactose, 2-deoxy-D-arabino-hexose, 2-deoxy-2,2-difluoro-D-arabino-hexose, 2-deoxy-2-fluoro-D-glucose 1-Phosphate, 2-deoxy-2-fluoro-D-glucose 6-P, 2-deoxy-2-fluoro-D-glucose 1,6 biphosphate, 2-deoxy-2-fluoro-D-mannose 1-P, 2-deoxy-2-fluoro-D-mannose 6-P, 2-deoxy-2-fluoro-D-mannose 1,6-biphosphate, nucleotide diphosphate (for example uridine di-P)-2deoxy-2-fluoro-D-glucose, mannose.

C-2-halogen substituted, and NH3 substituted derivatives of D-Glucose 6-phosphate, 2-deoxy-2-fluoro-2-D-glucose-6-phosphate, 2-chloro-2-deoxy-D-glucose-6-phosphate, 2-deoxy-D-arabino-hexose-6-phosphate, D-glucosamine-6-phosphate, 2-deoxy-2-fluoro-2-D-manose-6-P, and any known derivatives.

C-2 halogenated derivatives of hexose ring pyranoses (mannopyranoses, galactopyranoses), for instance C-2-deoxy-2-fluoro-D-pyranoses, and any known in the art.

Halogenated (fluoro, bromo, chloro, iodo) C2 sugars derivatives such as gluconolactones, glucuronic acid, glucopyranoside, and their phosphate derivatives.

Modification at C-1 or C-5: replacement of hydroxyl by fluorine or deoxygenation or replacement by a sulfur group in C-5, such as but not limited to glucosyl fluoride, 1-deoxy-D-glucose, 5-thio-D-glucose.

6-aminonicotinamide (6AN), indirectly by the inhibition of the PPP.

Inhibitors of phosphofructokinase (or fructose-6-P kinase) are any known in the art and may include, but are not limited to any of the following:
Acidosis-inducing agents, 2-deoxy-2-fluoro-D-glucose, citrate and halogenated derivatives of citrate, fructose 2,6-bisphosphate, bromoacetylethanolamine phosphate analogues (N-(2-methoxyethyl)-bromoacetamide, N-(2-ethoxyethyl)-bromoacetamide, N-(3-methoxypropyl)-bromoacetamide).

Inhibitors of aldolase: Analogs blocking the aldolase cleavage, thus blocking formation of trioses from fructose 1,6-bisphosphate require the presence of hydroxyl groups at C-3 and C-4. Thus, for example, 3-deoxy or 3-fluoro-D-glucose or 4-deoxy or 4-fluoro-D-glucose can be transformed to 4-fluoro-D-fructose 1,6-bisphosphate, which will not be cleaved by aldolase but will block it.

Inhibitors of glyceraldehyde 3P deshydrogenase are any known in the art and may include, but are not limited to any of the following:

Iodoacetate, pentalenolactone, arsenic, 1,1-difluoro-3-phosphate-glycerol.

Inhibitors of the transformation chain of glyceraldehyde (glyceraldehyde 3P deshydrogenase, phosphoglycerate kinase, phosphoglycerate mutase, enolase) are any which act at any step where phosphorylation is involved. Such inhibitors are any known in the art and may include, but are not limited to any of the following: either 2-fluoro (or iodo, or thio, or methoxy) or 3-fluoro (or 3,3 difluoro, 3-iodo, 3-carboxylo-, 3-thio)-glyceraldehydes or glycerates, 3-fluoro-2-phosphoglycerate, also, phosphothioesters or other phosphorous-modified analogs can block the transformations of glyceraldehyde.

Inhibitors of pyruvate kinase are any known in the art. Alternatively, a composition comprising serine or fructose 1,6-diP shifts the glycolytic pathway towards the TCA cycle; thus a composition of the invention comprises serine and an inhibitor of the TCA such as fluoroacetate or an inhibitor of the oxidative phosphorylation such as rhodamine.

Inhibitors of pyruvate carboxylase and PEP carboxylase, triose phosphate isomerase, phosphoglycerate kinase, enolase, phosphoglycerate mutase and triose phosphate isomerase are any known in the art.

Inhibitors of lactate deshydrogenase are any known in the art and may include, but are not limited to oxamate, 2-fluoro-propionic acid or it salts; 2,2-difluoro-propionic acid, pyruvate modified at C-3 such as, but not limited to 3-halo-pyruvate, 3-halopropionic acid and 2-thiomethylacetic acid.

Preferably, an inhibitor of glycolysis is any of 2FDG, oxamate and iodoacetate.

Glycolysis is the main the pathway for anaerobic ATP synthesis. Tumours switch to anaerobic ATP synthesis by metabolizing the well-distributed glucose among others in order to provide nucleotides through the PPP pathway. It is known that proliferating masses which are partly under anaerobic type respiration are more resistant to radiation or chemotherapy. Therefore, by locally inhibiting the glycolysis pathway, anaerobic respiration which is the principal energy pathway of poorly oxygenated cells is inhibited, leading to increased cell death of hypoxic proliferating cells. The proliferation of non-hypoxic cells is slowed as well owing to the shutdown of this primary energy pathway.

It may be possible to select which pathway is better to be shut down by performing a 2-FDG and a 11C-acetate positron emission tomography examination, and evaluating the activity of glycolysis and TCA cycles in the said tumour, allowing to choose for each individual tumour which compound should be favoured for the inhibition (e.g. more emphasis on glycolysis, more emphasis on TCA or both).

TCA Cycle Inhibitors

An example of a pathway involved in aerobic ATP synthesis is the tricarboxylic acid, TCA cycle (Krebs cycle). Enzymes associated with this pathway are known in the art and include pyruvate dehydrogenase complex, citrate synthase, aconitase, isocitrate lyase, alpha-ketoglutarate dehydrogenase complex, succinyl CoA synthetase, succinate dehydrogenase, fumarase, malate synthase, malate dehydrogenase, glutaminase and glutamate dehydrogenase (the later 2 indirectly). It is an aspect of the invention that an inhibitor of aerobic ATP synthesis is an inhibitor of an enzyme associated with the TCA cycle. Inhibitors of the TCA cycle are any known in the art.

General inhibitors of TCA: The availability of reduced and oxidized forms of nicotinamide adenine dinucleotide (NAD+ and NADH) is important for the TCA and depletors of NAD+ and NADH+would be inhibitors of the TCA cycle. Depletors of NAD⁺ and/or NADH include Hypoglycin A and its metabolite methylenecyclopropylacetic acid, ketone bodies (D(−)-3-hydroxybutyrate), alloxan, PNU and any other substance known in the art.

Inhibitors of pyruvate dehydrogenase are any known in the art and may include, but are not limited to any of the following:

Halo-pyruvate (e.g. 3-fluoropyruvate, 3-chloropyruvate, 3-bromopyruvate, 3-iodopyruvate)

Compounds of the general formula:
X—CH₂—CO—COOH
wherein X represents a halide, a sulphonate, a carboxylate, an alkoxide, or an amine oxide.

X may be a halide selected from the group consisting of fluoride, bromide, chloride and iodide.

X may be a sulphonate selected from the group consisting of triflate, mesylate and tosylate. X may be an amine oxide that is dimethylamine oxide.

Compounds of the general formula: 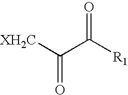
wherein X represents a halide, a sulphonate, a carboxylate, an alkoxide, or an amine oxide.

X may be a halide selected from the group consisting of fluoride, bromide, chloride and iodide.

X may be a sulphonate selected from the group consisting of triflate, mesylate and tosylate. X may be an amine oxide that is dimethylamine oxide.

R₁ may be OR, H, N(R″)2, C1-C6 alkyl, C6-C12 aryl, C1-C6 heteroalkyl, or a C6-C12 heteroaryl. Independently, R″ may represent H, C1-C6 alkyl, or C6-C12 aryl. Independently, R may be H, alkali metal, C1-C6 alkyl, C6-C12 aryl or C(O)R′; and R′ may represent H, C1-C20 alkyl or C6-C12 aryl.

Other inhibitors are arsenite, dichlorovinyl-cysteine, p-benzoquinone, thiaminase and any others known in the art.

Inhibitors of citrate synthetase are any known in the art and may include, but are not limited to any of the following:

Fluoroacetate (an its derivative fluoroacetyl-CoA), any halogenated acetyl-CoA, fluoroacetamide, fluorocrotonate, halogenated ketone bodies (for instance, chloroacetoacetate, fluoroacetoacetate, fluorohydroxybutyrate, chlorohydroxybutyrate, bromohydroxybutyrate), halogenated acetone, halogenated acetic acid (for example chloracetic acid), halogenated oleate (an analogue of ketone bodies) and any known in the art.

Inhibitors of aconitase are any known in the art and may include, but are not limited to any of the following:

Fluorocitrate, fluorocitrate 2R, 3R, and any other halogenated citrate (bromocitrate, chlorocitrate).

Inhibitors of isocitrate dehydrogenase are any known in the art and may include, but are not limited to any of the following:

DCVC (dichlorovinyl-cysteine)

Inhibitors of succinate dehydrogenase are any known in the art and may include, but are not limited to malonate, DCVC, Pentachlorobutadienyl-cysteine (or PCBD-cys), 2-bromohydroquinone, 3-nitropropionic acid, cis-crotonalide fungicides.

Inhibitors of succinyl CoA synthetase, alpha ketoglutarate dehydrogenase complex, fumarate hydratase (fumarase), malate dehydrogenase are any known in the art.

Inhibitors of glutaminase are any known in the art and may include, but are not limited to 6-diazo-5-oxo-L-norleucine (DON).

Inhibitors of glutamate dehydrogenase are any known in the art.

Other inhibitors of the TCA cycle include glu-hydroxyoxamate, p-chloromercuriphenylsulphonic acid (impermeant thiol agent), L-glutamate gamma-hydroxamate, p-chloromercuriphenylsulphonic acid, acivicin (alpha-amino-3-chloro-4,5-dihydro-5-isoxazoleacetic acid), halogenated glutamine and glutamate.

According to another embodiment of the invention, a TCA cycle inhibitor is any of arsenite, hypoglycin A, methylenecyclopropylacetic acid, alloxan, PNU, p-benzoquinone, fluoroacetate, halogenated acetates (iodo-, bromo-, chloro-acetate), halogenated acetyl-CoA (fluoroacetyl-CoA, bromoacetyl-CoA, chloroacetyl-CoA, iodoacetyl-CoA), halogenated crotonate (fluoro-, iodo-, bromo-, chloro-crotonate), halogenated ketone bodies, (chloro-, fluoro-, bromo-, iodoaceto-acetate, fluoro-, chloro-, bromo-, iodo-butyrate, fluoro-, chloro-, bromo-, iodo-acetone), halogenated oleate (iodo, bromo, chloro, fluoro-oleate), halogenated citrate, halogenated citrate 2R, 3R isomer (fluoro-, bromo-, chloro-, iodo-citrate), dichlorovinyl-cysteine, halogenated aminoacids, malonate, pentachlorobutadienyl-cysteine, 2-bromohydroquinone, 3-nitropropionic acid, cis-crotonalide fungicides, glu-hydroxyoxamate, p-chloromercuriphenylsulphonic acid, L-glutamate gamma-hydroxamate, p-chloromercuriphenylsulphonic acid, acivicin (alpha-amino-3-chloro-4,5-dihydro-5-isoxazoleacetic acid, halogenated glutamine (fluoro, iodo, chloro, bromo-glutamine), or halogenated glutamate (fluoro, iodo, chloro, bromo-glutamate).

Where more than one inhibitor of the TCA is present in a composition, preferably, one inhibitor is directed towards the upper half of the TCA cycle, which is characterised by providing no redox products such as NADH, HANPH, or FADH₂ (e.g. enzymes pyruvate dehydrogenase, citrate synthase, aconitase) and another inhibitor is directed towards the lower half of the TCA cycle, which is characterised by providing redox products such as NADH, HANPH, or FADH₂ (e.g. enzymes isocitrate lyase, alpha-ketoglutarate dehydrogenase complex, succinyl CoA synthetase, succinate dehydrogenase, malate synthase, glutaminase). Examples of a combination of inhibitor includes fluoroactetate and malonate.

Fluorocitrate and Derivatives

According to a preferred embodiment of the invention, a TCA cycle inhibitor of the invention has a formula (I): 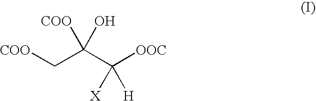
where X may be halide, a sulfonate, a carboxylate, an alkoxide, an amine oxide or a OH. The halide may be selected from the group consisting of: fluoride, bromide, chloride, and iodide. The sulfonate may be selected from the group consisting of: triflate, mesylate and tosylate. The carboxylate may be selected from the group consisting of: methoxylate and ethyloxylate. The alkoxide may be selected from the group consisting of: methoxide and ethoxide. The amine oxide is dimethylamine oxide. According to one aspect of the invention, the stereochemistry is 2R, 3R.
Fluoroacetate and Derivatives

TCA cycle inhibitors also includes substances which are converted into inhibitors of the TCA cycle such as, for example fluoroacetate and derivatives. According to a preferred embodiment of the invention, a TCA cycle inhibitor of the invention has a formula (II): 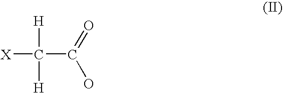
where X may be halide, a sulfonate, a carboxylate, an alkoxide, or an amine oxide, a OH.

The halide may be selected from the group consisting of: fluoride, bromide, chloride, and iodide. The sulfonate may be selected from the group consisting of: triflate, mesylate and tosylate. The carboxylate may be selected from the group consisting of: methoxylate and ethyloxylate. The alkoxide may be selected from the group consisting of: methoxide and ethoxide. The amine oxide may be dimethylamine oxide.

Several other compounds may block the production of ATP at the level of the TCA cycle after compound transformation. Indeed, most amino-acids may be degraded to enter the TCA cycle at various places. Therefore, most of aminoacids used in an halogenated formulation will be able to block the TCA cycle by being degraded to one of the TCA products (halogenated glutamate, glutamine, histidine, proline, arginine, valine, methionine, threonine, isoleucine, aspartate, tyrosine, phenylalanine, asparagine, aspartate, alanine, glycine, cysteine, serine, threonine). Some of the amino-acids will be transformed into ketone bodies. These amino-acids (leucine, lysine, phenylalanine, tyrosine) in an halogenated presentation will interact at the same sites where halogenated ketone bodies interact as described previously. Finally, some amino-acids in an halogenated formulation (tryptophan, leucine, isoleucine) will be directly transformed into acetyl-CoA and will block the TCA at the level of citrate synthase-aconitase.

Preferably, an inhibitor of the TCA cycle is any of fluoroacetate, fluorocitrate, bromopyruvate, arsenite, acetoacetate, and betahydroxybutyrate.

The requirement for both energy and building blocks such as lipids, amino acids, nucleic acids by rapidly proliferating cells means the TCA cycle is highly active. The inventors have found that the cells become susceptible to uptake of substrates of the TCA cycle such as acetate, much more so than non-proliferating cells. Therefore, treatment of readily proliferating cells with inhibitors of the TCA cycle benefits from rapid and selective inhibitor uptake, and cell death owing to the inhibition of the central anabolic pathway. More efficient cell death may be achieved in combination with other energy-producing pathways such as glycolysis and oxidative phosphorylation. Simultaneous inhibition of the PPP also shuts down an important anabolic pathway. It may be possible to select which pathway is better to inhibit by performing a 2-FDG and a ¹¹C-acetate positron emission tomography examination, and evaluating the activity of glycolysis and TCA cycles in the said tumour, allowing to choose for each individual tumour which compound should be favoured for the inhibition.

Simultaneous Inhibition of Other Pathways

According to an aspect of the invention a TCA cycle inhibitor is capable of inhibiting at least 3 cellular mechanisms of proliferating cells simultaneously. This may be achieved by blocking, for example, aconitase from the TCA cycle. The inventors have realised that the use of an aconitase inhibitor such as, for example, fluorocitrate (or fluoroacetate which is later converted into fluorocitrate) can inhibit other important pathways such as fatty acid synthesis at the level of ATP-citrate lyase and calcium intracellular signalling through derivatives accumulation.

Oxidative Phosphorylation

An example of a pathway involved in aerobic ATP synthesis is oxidative phosphorylation. Enzymes associated with this pathway are known in the art and include enzyme complex I (NADH coenzyme Q reductase), II (succinate-coenzyme Q reductase), III (coenzyme Q cytochrome C reductase), IV (cytochrome oxydase), and V (F0-F1, ATP synthase). It is an aspect of the invention that an inhibitor of aerobic ATP synthesis is an inhibitor of an enzyme associated with oxidative phosphorylation.

Inhibitors of enzyme complex I are any known in the art and may include, but are not limited to any of the following: tritylthioalanine, caminomycin, and piperazinedione, rotenone, amytal, 1-methyl-4-phenylpyridinium (MPP+), paraquat, methylene blue, Ferricyanide (the later 2 are electron acceptors).

Inhibitors of enzyme complex II are any known in the art.

Inhibitors of coenzyme Q are any known in the art.

Inhibitors of enzyme complex III are any known in the art and may include, but are not limited to myxothiazol, antimycin A, ubisemiquinone, cytochrome C, 4,6-diaminotriazine derivatives, metothrexate or electron acceptors such as phenazine methosulfate and 2,6-Dichlorophenol-indophenol.

Inhibitors of enzyme complex IV are any known in the art and may include, but are not limited to cyanide, hydrogen sulfide, azide, formate, phosphine, carbon monoxide and electon acceptor ferricyanide.

Inhibitors of enzyme complex V are any known in the art and may include, but are not limited to VM-26 (4′-demethyl-epipodophyllotoxin thenylidene glucoside), tritylthioalanine, caminomycin, piperazinedione, dinitrophenol, dinitrocresol, 2-hydroxy-3-alkyl-1,4-naphtoquinones, apoptolidin aglycone, oligomycin, ossamycin, cytovaricin, naphtoquinone derivatives (e.g. dichloroallyl-lawsone and lapachol), rhodamine, rhodamine 123, rhodamine 6G, carbonyl cyanide p-trifluoromethoxyphenylhydrazone, valinomycin, rothenone, safranine O, cyhexatin, DDT, chlordecone, arsenate, pentachlorophenol, benzonitrile, thiadiazole herbicides, salicylate, cationic amphilic drugs (amiodarone, perhexyline), gramicidin, calcimycin, pentachlorobutadienyl-cysteine (PCBD-cys), trifluorocarbonylcyanide phenylhydrazone (FCCP).

Where rhodamine, rhodamine 123, or rhodamine 6G are present in the composition, they may be used as direct inhibitors of oxidative phosphorylation, and not as a dye for homogeneous coating control, or for photodynamic therapy, for instance. Therefore, where rhodamine compounds are used, the treatment according to the invention is not in combination with rhodamine based imaging or light based treatment.

Other inhibitors of oxidative phorphorylation may include atractyloside, DDT, free fatty acids, lysophospholipids, n-ethylmaleimide, mersanyl, p-benzoquinone.

Preferably, an inhibitor of oxidative phosphorylation is any of rhodamine, dinitrophenol, and rotenone.

Inhibition of oxidative phosphorylation surprisingly leads to an inhibition of proliferation; proliferating cells do not obtain energy via other pathways because the final phases of the most important ATP production in a cell is concentrated in mitochondria. The inventors have also found that inhibition of both oxidative phosphorylation and glycolysis is further effective against proliferating cells. This might be explained by the fact that anaerobic type cancer cells are not efficient in ATP production, and much less efficient than well oxygenated cells. These cells heavily depend on the non efficient glycolytic pathway. Therefore, an uncoupling of the oxidative phosphorylation added to the anaerobic inefficiency creates even more dependence on glycolytic ATP production. The interruption of glycolysis in these conditions leads to accelerated cell death.

Because inhibitors of oxidative phosphorylation are toxic to a subject in the doses needed for efficacy via the systemic route, such inhibitors have been largely overlooked for effective treatment of conditions such as cancer. For example, it is known that the LD10 (the dose which kills 10% of animals) for rhodamine 123 in rodents is 20 mg/kg. It is also known that the maximal tolerated dose of Rhodamine 123 delivered intravenously in man is 95 mg/m². For a 70 kg patient, the tolerated dose of systemically delivered rhodamine is about 150 mg for each injection. Operating at these maximum, non-lethal doses, a clinical trial did not shown any significant effect on prostate tumours, although rhodamine accumulation was confirmed in the prostate of the patients.

By locally delivering oxidative phosphorylation inhibitors on an implant, the delivery period is prolonged i.e. the inhibitors are not cleared by the liver, and the dose received by the tumour is higher with intra-venous infusion. Furthermore, the lethal dose can be greatly exceeded. For instance delivering 40 or 50 mg of rhodamine leads to tumour death, and not just to a slow down in tumour growth. Such a dose, delivered systemically, would require 100 g of rhodamine, more than 1000 times the lethal dose. Thus, an implant coated with an oxidative phosphorylation inhibitor increases the effective dose to the tumour or to the tumour resection cavity and permits greater than lethal dosing.

Furthermore, the oxidative phosphorylation inhibitor coated implant may be used in combination with radiotherapy, where a lower dose of inhibitor may be used.

Furthermore, the oxidative phosphorylation inhibitor coated implant may be used in combination with chemotherapy, where a lower dose of inhibitor may be used.

Pentose Phosphate Pathway Inhibitors

Enzymes associated with the pentose phosphate pathway are known in the art and include glucose-6-phosphate dehydrogenase, lactonase, 6-phosphogluconate dehydrogenase, phosphopentose isomerase, phosphopentose epimerase, transketolase, and transaldolase.

Inhibitors of glucose 6P dehydrogenase are any known in the art and may include, but are not limited to any of the following halogenated (fluorinated), D-hexoses (e.g. 2-Amino-2-deoxy-D-glucose-6-phosphate (D-glucosamine-6-phosphate)).

Inhibitors of lactonase are any known in the art.

Inhibitors of 6P gluconate dehydrogenase are any known in the art and may include, but are not limited to 6-aminonicotinamide (6AN).

Inhibitors of phosphopentose isomerase, phosphopentose epimerase, transketolase, transaldolase are any known in the art.

The pentose phosphate pathway (PPP) starts with the conversion of glucose-6-phosphate to ribose-5-phosphate; the former is obtained from the first step of glycolysis. Glycolysis accounts for a small fraction of the energy requirements of the aerobically respiring cell, the majority being obtained from oxidative phosphorylation. Despite this, the glycolytic pathway is highly active in proliferating cell masses. Glycolysis is believed to act as an anabolic pathway, by providing the starting substrate for the PPP, for the ultimate synthesis of NADPH. The latter is used for biosynthetic purposes, and is highly active in proliferating cell masses. Consequently inhibition of oxidative phosphorylation pathways has the effect of inhibiting cell growth by inhibiting the supply of substrates (NADPH) to the anabolic pathway.

Individual and Combinations of Inhibitors

According to one aspect the invention, a composition comprises one or more inhibitors of aerobic ATP synthesis (e.g. inhibitors of TCA and/or oxidative phosphorylation or oxphos). According to one aspect the invention, a composition comprises one or more inhibitors of aerobic ATP synthesis (e.g. inhibitors of TCA and/or oxidative phosphorylation or oxphos) combined with inhibitors of the PPP. According to another aspect of the invention, a composition comprises one or more inhibitors of anaerobic ATP synthesis (e.g. inhibitors of the glycolysis directly, or indirectly through inhibition of PPP).

Owing to the properties of proliferating cells, the inventors find that a composition comprising combinations of inhibitors may also be effective at reducing a rapidly proliferating cell mass.

Owing to the properties of proliferating cells, the inventors find that a composition comprising combinations of inhibitors may also be effective in the treatment of the lymphatic pathways (drainage of the tumour or tumour cavity through the lymphatic vessels), in order to treat the cells that have gone in these vessels and in the lymph nodes. It is an elegant fashion to treat microscopic lymphatic vessel and node invasion.

According to another aspect of the invention, a composition comprises one or more inhibitors of aerobic ATP synthesis and one or more inhibitors of the PPP. According to one aspect of the invention a combination of inhibitors in such a composition may be one or more inhibitors of TCA and/or oxidative phosphorylation and one or more inhibitors of the PPP. An inhibitor of TCA may be, for example, fluoroacetate or malonate, an inhibitor of oxphos may be oligomycin or rhodamine, and an inhibitor of the PPP may be 6-aminonicotinamide (which in the end blocks glycolysis).

According to another aspect of the invention, a composition comprises one or more inhibitors of aerobic and one or more inhibitors of anaerobic ATP synthesis. A combination may be, for example, one or more inhibitors of glycolysis and one or more inhibitors of the TCA cycle. In such case, inhibitors may be 2FDG or iodoacetate or oxamate (glycolysis) and fluoroacetate or fluorohydroxybutyrate (TCA cycle). An alternative combination may be, for example, one or more inhibitors of glycolysis and one or more inhibitors of oxidative phosphorylation. In such case, inhibitors may be 2FDG or iodoacetate or oxamate (glycolysis) and rhodamine or dinitrophenol (oxidative phosphorylation). Yet another alternative combination may be, for example, one or more inhibitors of glycolysis, one or more inhibitors of the TCA cycle and one or more inhibitors of oxidative phosphorylation. In such case, inhibitors may be 2FDG or iodoacetate or oxamate (glycolysis), fluoroacetate or fluorohydroxybutyrate (TCA cycle) and rhodamine or dinitrophenol (oxidative phosphorylation).

According to another aspect of the invention, a composition comprises one or more inhibitors of aerobic ATP synthesis, and one or more inhibitors of anaerobic ATP synthesis and/or one or more inhibitors of the PPP. Combinations of inhibited pathways include (a) TCA cycle (aerobic) and PPP, (b) oxidative phosphorylation (aerobic) and PPP, (c) glycolysis (anaerobic), TCA cycle (aerobic), oxidative phosphorylation (aerobic) and PPP. In such cases combinations of inhibitors include, for example (a) fluoroacetate and 6-aminonicotinamide, (b) rhodamine and 6-aminonicotinamide, and (c) 2FDG, fluoroacetate, rhodamine and 6-aminonicotinamide.

According to another aspect of the invention, a composition comprises one or more inhibitors active against both aerobic and anaerobic ATP synthesis simultaneously. Examples of such inhibitors include, but are not limited to arsenite (blocks both glycolysis and oxphos), fluoroacetate (which blocks TCA and, as a consequence of fluorocitrate accumulation it blocks glycolysis), fluoroacetoacetate, which blocks TCA and glycolysis.

It is an aspect of the invention that the composition is formulated to primarily inhibit the glycolytic pathway (possibly through PPP inhibition) and secondarily inhibit either the TCA cycle or oxidative phosphorylation. Such composition is formulated according to the dose and efficacy of the inhibitory compounds.

The inventors have advantageously found that by inhibiting a combination of ATP synthetic pathways, optionally in combination with the inhibition of the PPP, the synthesis of ATP in proliferating cell masses may be most effectively inhibited, leading to rapid and selective tumour shrinkage. The combination of ATP inhibitors is very important for the inventors. The inventor's own clinical experience (MR spectroscopy and PET-CT examinations) as well as data from the literature confirm the great variety of substrates taken up by tumours. A review of the literature data shows for instance that the affinity for 18-FDG intake varies from 3 to 100%, depending on evaluated tumours and affected organs (e.g. http://www.petscaninfo.com/zportal/portals/phys/clinical/jnmpetlit/index_html/JNM_Onco Apps/JNM_Table8/article_elements_view). The inventors have also observed that a beneficial therapeutic approach is to treat several ATP synthesis pathways simultaneously.

Derivatives

Stereoisomer, tautomers, racemates, prodrugs, metabolites, pharmaceutically acceptable salts, bases, esters, structurally related compounds or solvates of the ATP synthesis inhibitors are within the scope of the invention.

The pharmaceutically acceptable salts of the inhibitors according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such a sarginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

The term “stereoisomer”, as used herein, defines all possible compounds made up of the same atoms bonded by the same sequence of bonds but having different three-dimensional structures which are not interchangeable, which the inhibitors of the present invention may possess. Unless otherwise mentioned or indicated, the chemical designation of an inhibitor herein encompasses the mixture of all possible stereochemically isomeric forms, which said compound may possess. Said mixture may contain all diastereomers and/or enantiomers of the basic molecular structure of said compound. All stereochemically isomeric forms of the inhibitors of the invention either in pure form or in admixture with each other are intended to fall within the scope of the present invention.

The inhibitors according to the invention may also exist in their tautomeric forms. Such forms, although not explicitly indicated in the inhibitors described herein, are intended to be included within the scope of the present invention.

For therapeutic use, the salts of the inhibitors according to the invention are those wherein the counter-ion is pharmaceutically or physiologically acceptable.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo. Specific examples of prodrugs comprising cholesterol or vitamin A are described below.

Slow Release Formulation

Described above is the provision of a top coat for regulating the release of inhibitory compounds. Another aspect of the invention relates to a composition comprising additives which control inhibitor release. According to another embodiment of the invention, the composition is a slow release formulation. Accordingly, the implant may be provided with a large or concentrated dose of inhibitor. Once the implant is at the site of treatment, inhibitor is released at a rate determined by the formulation. This avoids the need for frequently replacing implants to maintain a particular dose. Another advantage of a slow release formulation is that the composition diffuses day and night, over several days or weeks. Furthermore, the present inventors have found that inhibitor uptake can be relatively slow in some tumours by observing ¹⁸F-FDG and ¹¹C-acetate by PET-CT. Although tumours may show as an intense signal due to the sensitivity of the imaging and probe, this is deceptive of the uptake rate which can be relative low e.g in the range of ng/min. Consequently, a slowly releasing inhibitor is better able to match the rate of inhibitor take by the tumour, and avoid wasteful and toxic overdosing.

One embodiment of the present invention is an implant comprising a composition as described herein, wherein said composition further comprises one or more slow release agents. Slow release agents may be natural or synthetic polymers, or reabsorbable systems such as magnesium alloys.

Among the synthetic polymers useful according to a slow release formulation of the invention are poly(glycolic) acid, poly(lactic acid) or in general glycolic- and lactic acid based polymers and copolymers. They also include poly caprolactones and in general, poly hydroxyl alkanoates (PHAs) (poly(hydroxy alcanoic acids)=all polyester). They also include Poly(ethylene glycol), poly vinyl alcohol, poly(orthoesters), poly (anhydrides), poly(carbonates), poly amides, poly imides, poly imines, poly(imino carbonates), poly(ethylene imines), polydioxanes, poly oxyethylene (poly ethylene oxide), poly(phosphazenes), poly sulphones, lipids, poly acrylic acids, poly methylmethacrylate (PMMA), poly acryl amides, poly acrylo nitriles (Poly cyano acrylates), poly HEMA, poly urethanes, poly olefins, poly styrene, poly terephthalates, poly ethylenes, poly propylenes, poly ether ketones, poly vinylchlorides, poly fluorides, silicones, poly silicates (bioactive glass). siloxanes (Poly dimethyl siloxanes), hydroxyapatites, lactide-capronolactone, and any other synthetic polymer known to a person skilled in the art. Other synthetic polymers may be made from hydrogels based on activated polyethyleneglycols (PEGs) combined with alkaline hydrolyzed animal or vegetal proteins.

Among the natural derived polymers useful according to a slow release formulation of the invention, are poly aminoacids (natural and non natural), poly β-aminoesters. They also include poly(peptides) such as: albumines, alginates, cellulose/cellulose acetates, chitin/chitosan, coliagene, fibrine/fibrinogen, gelatine, lignine. In general, proteine based polymers. Poly(lysine), poly(glutamate), poly(malonates), poly(hyaluronic acids). Poly nucleic acids, poly saccharides, poly(hydroxyalkanoates), poly isoprenoids, starch based polymers, and any other natural derived polymer known to a person skilled in the art.

For both synthetic and natural polymers, the invention includes copolymers thereof are included as well, such as linear, branched, hyperbranched, dendrimers, crosslinked, functionalised (surface, functional groups, hydrophilic/hydrophobic).

The slow release composition may be formulated as liquids or semi-liquids, such as solutions, gels, hydrogels, suspensions, lattices, liposomes. Any suitable formulation known to the skilled man is within the scope the scope of the invention. According to an aspect of the invention, a composition is formulated such that the quantity of inhibitor is between less than 1% and 60% of total slow-release polymer mass. According to an aspect of the invention, a composition is formulated such that the quantity of inhibitor is between 1% and 50%, 1% and 40%, 1% and 30%, 1% and 20%, 2% and 60%, 5% and 60%, 10% and 60%, 20% and 60%, 30% and 60%, or 40% and 60% of total slow-release polymer mass.

Solubilising Agents

According to another embodiment of the present invention, the composition comprises at least one inhibitor of ATP synthesis as described herein coupled to one or more solubilising agents. Such agents change the hydrophilic and hydrophobic profile of the inhibitor, depending on the required solubility. For example, if a composition according to the invention comprises a hydrophilic TCA cycle inhibitor such as fluoroacetate, and a hydrophobic slow release polymer such as polyorthoester, the inhibitor will not adequately suspend within the composition. Similarly, a composition according to the invention comprising a very hydrophilic oxidative phosphorylation inhibitor such as rhodamine 123 and polyorthoester slow release agent, will lead to an inadequately emulisified composition. Consequently the release properties of the slow release agent may be compromised, and degradation within the body accelerated. To overcome this, the inventors have coupled at least one ATP synthesis inhibitor a solubilising agent which changes the hydrophobicity or hydrophilicity of the inhibitor, depending on the required formulation. The composition so formed is more stable. According to one aspect of the invention, the coupled compound is a prodrug wherein the solubilising agent is cleaved in vivo, so releasing the inhibitor. According to another aspect of the invention, the solubilising agent is cleaved from the inhibitor more rapidly by the proliferating cells.

Cholesterol

According to one aspect of the invention, cholesterol (II) or a derivative thereof is a solubilising agent. One embodiment of the invention is an implant provided with a composition as mentioned above in which at least one ATP synthesis inhibitor as described herein is coupled to cholesterol (III) or derivatives thereof: 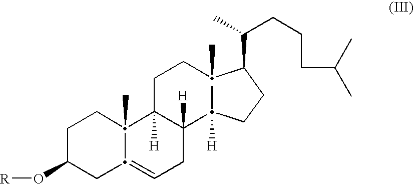
wherein R may be one of the following substances: betahydroxybutyrate, halogenated butyrate, halogenated acetate, halogenated aceto-acetate, halogenated acetamide, halogenated crotonate, halogenated acetone, halogenated citrate and halogenated oleate.

Derivatives of cholesterol are modifications which retain or enhance of activity of the parent compound. Derivatives include, but are not limited to cholesteryl-3-betahydroxybutyrate, cholesteryl-halogenated butyrate, cholesteryl-halogenated acetate, cholesteryl-halogenated aceto-acetate, cholesteryl-halogenated acetamide, cholesteryl-halogenated crotonate, cholesteryl-halogenated acetone, cholesteryl-halogenated citrate, or cholesteryl-halogenated ofeate.

Halogenated means fluoro-, chloro-, bromo- or iodo-modified.

An advantage of using cholesterol or a derivative thereof as a solubilising agent is such natural metabolite can enter a cell via a number of mechanisms including through the lipid bilayer of the cell membrane. In rapidly proliferating cells, absorption is more rapid due to the requirement for cholesterol in cell membranes. Once in the lipid bilayer, flippase enzyme transfers the cholesterol-coupled inhibitor from the outer layer to the inner layer; cholesterol is internalised in the cytosol and the inhibitor is released from cholesterol by cholesterol-metabolising enzymes.

A cholesterol is coupled to an inhibitor using known methods. For example, coupling may proceed via nucleophilic attack by electrons of an oxygen atom on the cholesterol. For example, coupling may proceed via nucleophilic attack by electrons of an oxygen atom on the inhibitor. According to another example, esters, ethers or other derivatives of cholesterol or inhibitor may be prepared to facilitate coupling. Mechanisms and knowledge of appropriate coupling moieties are known to the skilled person for the preparation of such coupled inhibitors.

One embodiment of the present invention is the composition comprises cholesteryl-fluoroacetate and polyorthoester.

Vitamin A

According to one aspect of the invention, vitamin A (retinol) or a derivative thereof is a solubilising agent. One embodiment of the invention is an implant provided with a composition as mentioned above, wherein at least one ATP synthesis inhibitor is coupled to vitamin A or derivatives thereof. Examples of derivatives include the ether (IV) and ester (V) forms which groups facilitate ease of coupling:

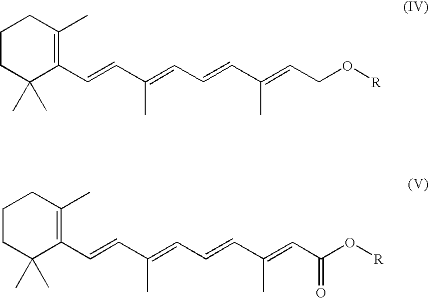

Wherein R may be one of the following substances: betahydroxybutyrate, halogenated butyrate, halogenated acetate, halogenated aceto-acetate, halogenated acetamide, halogenated crotonate, halogenated acetone, halogenated citrate, halogenated oleate.

Derivatives of vitamin A are modifications which retain or enhance of activity of the parent compound. Derivatives include, but are not limited to those mentioned above for the inhibitors and betahydroxybutyrate, halogenated butyrate, halogenated acetate, halogenated aceto-acetate, halogenated acetamide, halogenated crotonate, halogenated acetone, halogenated citrate, or halogenated oleate.

Halogenated means fluoro-, chloro-, bromo- or iodo-modified.

An advantage of using vitamin A or a derivative thereof as a solubilising agent is that such natural metabolite can enter a cell via a number of mechanisms. In rapidly proliferating cells, absorption is more rapid, especially in vitamin A metabolising cells such as found in liver tissue. The effect may be used to treat, for instance, hepatocarcinomas by injecting a slow release polymer of retinyl ether or retinoic acids ester coupled with haloacetates directly inside the hepatpcarcinoma mass. The antiproliferative effect commences once the inhibitor is liberated from the polymer and vitamin A is metabolised.

A vitamin A is coupled to an inhibitor using known methods. For example, coupling may proceed via nucleophilic attack by electrons of an oxygen atom on the vitamin A. For example, coupling may proceed via nucleophilic attack by electrons of an oxygen atom on the inhibitor. According to another example, esters or other derivatives of vitamin A or inhibitor may be prepared to facilitate coupling. Mechanisms and knowledge of active groups are known to the skilled person for the preparation of such coupled inhibitors

According to one embodiment of the present invention, the composition comprises vitamin A-fluoroacetate and polyorthoester.

Bone Metastases

Another embodiment of the present invention is a composition as described herein further comprising pyrophosphate. Said composition may be used to treat a bone tumour first, by preventing tumour cell proliferation, and in a second step, to stimulate bone reconstruction. The composition may be provided on a bioabsorbable implant for insertion into a bone metastasis or cavity left after removal of a bone cancer. According to the present invention pyrophosphate may be any suitable salt of pyrophosphate, including, but not limited to sodium pyrophosphate, potassium pyrophosphate, calcium pyrophosphate. The pyrophosphates may be mixed to the polymer containing the inhibitors. Once the polymer has been degraded and all inhibitors have been absorbed, the presence of pyrophosphates may stimulate new bone formation.

Encapsulated Inhibitor

According to one aspect of the invention at least one ATP synthesis inhibitor described herein is encapsulated in one or more micro-capsules or nano-capsules.

Examples of nano-capsules (or nano-spheres) or formulations therewith include, but are not limited to a copolymer poly(ethylene oxide) with poly(L-Lactic acid) or with poly(beta-benzyl-L-aspartate); copolymer with poly(lactide-co-glycolide)-[(propylene oxide)-poly(ethylene oxide)]; polyphosphazene derivatives; poly(ethylene glycol) coated nanospheres; poly(isobutylcyanoacrylate) nanocapsules; poly(gamma-benzyl-L-glutamate)/(poly(ethylene oxide); chitosan-poly(ethylene oxide) nanoparticles; nanoparticles where the anti-proliferative drug is prepared using o-carboxymethylate chitosan (o-CMC) as wall forming material; silicone nanocapsules, solid lipid nanoparticles or nanospheres (SLNs) and any known formulation of nano-particles known to someone skilled in the art.

Examples of micro-capsules (or micro-spheres) or formulations therewith include but are not limited to multiporous beads of chitosan; coated alginate microspheres; N-(aminoalkyl) chitosan microspheres; chitosan/calcium alginate beads, poly(adipic anhydride) microspheres; gellan-gum beads; poly(D, L-lactide-co-glycolide) microspheres; alginate-poly-L-lysine microcapsules; crosslinked chitosan microspheres; chitosan/gelatin microspheres; crosslinked chitosan network beads with spacer groups; aliphatic polyesters such as 1,5-diozepan-2-one and D,L-dilactide microspheres; triglyceride lipospheres; polyelectrolyte complexes of sodium alginate chitosan; polypeptide microcapsules; albumin microspheres; and any other micro-capsule (or micro-sphere) formulation known to someone skilled in the art.

By using encapsulated inhibitor, the solubility profile of the inhibitor may be changed according to the environment of the formulation. It may thus act as a solubilising agent as mentioned above. An encapsulated inhibitor has an advantage that solubilisation does not require chemical coupling of the inhibitor. Inhibitors of ATP synthesis are generally hydrophilic while slow-release gels where present which are generally hydrophobic. Thus, an encapsulated inhibitor allows solubility within a slow-release gel (polymer), so preventing an otherwise unstable formulation.

Furthermore, encapsulation may be used to modulate release of the inhibitor (e.g. fine tune or prolong release time). Furthermore, encapsulation may be used to improve intracellular penetration, as known for encapsulations such as SLN. The advantages of encapsulated formulation may be applied to inhibitor already chemically modified to improve solubility. For example, cholesterol coupled fluoroacetate may be prepared in microcapsules within a slow release gel. The formulation so produced would provide solubility for the inhibitor, slow release modulated by the presence of capsules and active cellular penetration. Such composition may reduce the frequency and/or duration of treatment compare with conventional formulations.

One embodiment of the present invention is an implant provided with a composition as mentioned above in which at least one ATP synthesis inhibitor is encapsulated in micro- or nano-capsule(s) (or micro- or nano-sphere(s)). According to one aspect of the invention, at least one inhibitor is also pre-coupled to a solubilising agent as mentioned above.

Sensitising Agents

Another embodiment of the present invention is an implant provided with a composition as mentioned above, said composition further comprising one or more components to sensitise the proliferating cells to the inhibitors of the composition. It is achieved by unlocking or unblocking the flow into the TCA cycle. For example, where glycolysis is blocked at the level of pyruvate kinase it is possible to add serine or other elements (e.g. fructose 1-6 diP) to the composition. This forces the pyruvate kinase enzyme to adapt to its tetrameric active form and release the pyruvate kinase inhibition (Mazurek S, Lüftner D, Wechsel H W, Schneider J, Eigenbrodt E. Tumor M2-PK: a marker of the tumor metabolome, in Tumor markers: physiology, pathobiology, technology and clinical applications. Eleftherios P et al, AACC Press 2002, 471-475). The result is a stimulation of the TCA cycle. In cells where the TCA cycle is not very active, this lifts the inhibition, raises the TCA metabolism and, because of that, increases the sensitivity of the cells to TCA inhibitors.

Dose

The size of implant and concentration of composition thereon can be calculated using known techniques by the skilled person.

According to one aspect of the invention, an implant is coated with a composition comprising TCA inhibitor such that the inhibitor concentration delivered to a subject is greater than or equal to 1, 10, 20, 40, 60, 80, 100, 150, 200, 300, 400, 500, 600, 700, 800, 900, 1000 or mg inhibitor/kg, or a concentration in the range between any two of the aforementioned values. Preferably the dose is between 1 and 10 mg/kg.

According to one aspect of the invention, an implant is coated with a composition comprising glycolysis inhibitor such that the inhibitor concentration delivered to a subject is greater than or equal to 20, 40, 60, 80, 100, 150, 200, 300, 400, 500, 600, 700, 800, 900, 1000 or mg inhibitor/kg, or a concentration in the range between any two of the aforementioned values. Preferably the dose is between 20 and 400 mg/kg.

According to one aspect of the invention, an implant is coated with a composition comprising oxidative phosphorylation inhibitor such that the inhibitor concentration delivered to a subject is greater than or equal to 5, 10, 15, 20, 25, 30, 35, 40, 45, 50, 55 or 60 mg inhibitor/kg, or a concentration in the range between any two of the aforementioned values. Preferably the dose is between 10 and 60 mg/kg.

The quantity of inhibitor per mm² of implant required to arrive at the above doses can be readily calculated by the skilled person.

The case of fluoroacetate, for example, a typical loading might be 0.05-0.3 milligrams of fluoroacetate per square cm of implant surface for a breast implantation device.

The case of 2FDG, for example, a typical loading might be 10 to 30, 15 to 25, 18 to 22, or 20 milligrams of 2FDG per square cm of implant surface in order to deliver an equivalent dose of 400-800 mg/kg dose to the first cm of resection cavity wall. The loading may be recalculated in case a different dose is required, for example for aggressive cancer, to deliver 3 g/kg, the loading is calculated to be 100 milligram/square cm. Such high dosages are particularly relevant for the treatment of naive cancer patients or for the improved treatment of cancer resection cavity, possibly using an implant coated with a foil or a polymeric layer, allowing better surface contact of the implant surface with the area to treat. Such calculations are known to the skilled person. Once in place, for instance at the level of a brain resection cavity, the patient will benefit from chemotherapy and radiotherapy, with improved efficacy.

According to an aspect of the invention, a specification combination of loading comprises less than 10 micrograms of 2FDG per square mm of undeployed coronary implant surface, together with less than 1 microgram of fluoroacetate and/or less than 5 micrograms rhodamine per square mm of undeployed implant surface, or dinitrophenol less then 5 micrograms per square mm of undeployed implant surface.

The case of bromopyruvate, for example, a typical loading might be less than 3 micrograms of bromopyruvate per square mm of undeployed implant surface in order to deliver an equivalent dose of 400 mg/kg dose or less to the first mm of vessel wall depth. Such calculations are known to the skilled person.

The case of arsenite, for example, a typical loading might be less than 3 micrograms of arsenite per square mm of undeployed implant surface in order to deliver an equivalent dose of 400 mg/kg dose or less to the first mm of vessel wall depth. Such calculations are known to the skilled person.

The case of fluoroacetoacetate, for example, a typical loading might be less than 5 micrograms of fluoroacetoacetate per square mm of undeployed implant surface in order to deliver an equivalent dose of 400 mg/kg dose or less to the first mm of vessel wall depth. Such calculations are known to the skilled person.

The case of rhodamine, for example, a typical loading might be in the range of 1 to 5 mg of rhodamine per square cm of undeployed implant surface in order to deliver an equivalent dose of 20 mg/kg or less to the first mm of the cavity depth (20 mg/kg means 20 mg per kg of surrounding tissue, such as breast, muscle, etc). Such calculations are known to the skilled person. For a 1-5 mm layer of composition comprising a slow-release polymer deposited on the, this would represent 1 to 50% of polymer composition.

According to one aspect of the invention, an implant is coated with a composition comprising rhodamine to deliver a concentration in the range 30 to 50 mg/kg of the treated organ, 35 to 50, and preferably 40 to 50 mg/kg. According to another aspect of the invention, an implant is coated with a composition comprising rhodamine to deliver a concentration in the range 5 to 30 mg/kg, 10 to 30, and preferably 20 to 30 mg/kg of the treated organ, when used in combination with cytotoxic therapy such as chemo- or radiotherapy.

According to an aspect of the invention, a specification combination of loading comprises less than 10 mg of iodoacetate or oxamate per square cm of undeployed implant surface, together with less than 300 microgram of fluoroacetate or less than 100 microgram of fluorocitrate or less than 5 mg rhodamine, or less then 5 mg of dinitrophenol per square cm of implant surface.

The dose would be calculated according to the condition being treated. For example, a higher dose will be required for the treatment of cancer compared with benign cell proliferation. Furthermore, the dose may be lower when used in combination with other cytotoxic treatments such as chemotherapy or radiotherapy.

Kit

A kit according to the invention may comprise at least one implant and separately, at least one composition of the present invention. The kit enables a technician or other person to coat a implant with a composition prior to insertion into a cavity.

The composition, besides comprising at least one type of inhibitor of ATP synthesis, optionally at least one inhibitor of the PPP, may contain additional substances that facilitate the coating of the implant by the end-user. The composition may contain, for example, fast evaporating solvents so as to allow the rapid drying of the implant. It may contain polymeric material to allow the inhibitors to adhere to the implant and/or facilitate its slow release.

The composition may be applied to the implant of the kit by any means known in the art. For example, by dipping the implant in the composition, by spraying the implant with the composition, by using electrostatic forces. Such methods are known in the art.

It is an aspect of the invention that the composition is provided in a container. For example, a vial, a sachet, a screw-cap bottle, a syringe, a non-resealable vessel, a resealable vessel. Such containers are any that are suitable for containing a composition and optionally facilitating the application of the composition to the implant. Indeed, some polymers to be used for the coating and the controlled release of the active compound, such as polyorthoesthers, are extremely unstable, are very sensitive to humidity and should be conserved in a cold atmosphere and in an argon atmosphere for instance. Some active products as well, such as rotenone, are sensitive to light and heat and should be preserved in dark and cold. In such a case, a container with the composition is kept separately from the bare implant. The interventional cardiologist may open the box containing the coating and apply it on the implant just before the intervention.

A kit according to the invention may comprise at a least foil pre-disposed with composition. The kit enables a technician or other person to cover any balloon without the need to manually coat the balloon. A kit may comprise more than one type of implant and more than one container of composition. A kit may provide a range of implant sizes, implant configurations, implant made from different materials. A kit may provide a range of vials containing different compositions with different inhibitors, different combinations of inhibitors, different combinations of polymers. A kit may facilitate the sequential application of more than one type of composition. A kit may contain instructions for use.

Combined Radiotherapy or Chemotherapy Treatment

The inventors have found that the combination of an implant according to the present invention and radiotherapy and/or chemotherapy for the treatment of proliferating cells (e.g. cancerous tumours or tumour resectioned cavities) provides an effective combination therapy to shrink cellular proliferations and kill tumours or tumour cells. The use of the implant can lead to effective treatment using a fraction of the normal radiotherapy or chemotherapy therapeutic dose.

According to this aspect of the invention, a tumour is totally or partially resected and an implant is placed inside the resection cavity as mentioned above. In addition, the site of the tumour is treated with radiotherapy applied either from an exterior source, or by using an implant containing at least one catheter and treating the lumen from inside with brachytherapy. The combination of implant and radiotherapy treatment may lead to a rapid and effective shrinking or death of the residual tumour or tumour cells because it renders the tumour cells much more sensitive.

Alternatively, a tumour may be totally or partially resected and an implant placed inside the resection cavity as mentioned above and, in addition, the site of the tumour is treated with intravenous chemotherapy (for instance paclitaxel, cisplatinum, vinorelbine, etc). The combination of implant and radiotherapy and/or chemotherapy treatments may lead to a rapid and effective shrinking or death of the residual tumour or tumour cells. It is expected that chemotherapy and/or radiotherapy will be much more efficient after the local application of the inhibitors inside the proliferating process. It is foreseen that accumulated doses of radiotherapy and/or chemotherapy could be decreased by 10 to 50%.

One embodiment of the present invention is an implant as described herein for use in treating tumours, in combination with radiotherapy. Another embodiment of the present invention is a method for treating a tumour comprising the use of an implant as described herein in combination with radiotherapy. Another embodiment of the present invention is use of an implant for treating a tumour, in combination with radiotherapy. Another embodiment of the present invention is an implant as described herein for use in treating tumours, in combination with chemotherapy. Another embodiment of the present invention is a method for treating a tumour comprising the use of an implant as described herein in combination with chemotherapy. Another embodiment of the present invention is use of an implant for treating a tumour, in combination with chemotherapy.

Another embodiment of the present invention is a method for reducing the dose of radiotherapy treatment of a tumour, comprising applying an implant in the vicinity of said tumour prior to radiotherapy.

Another embodiment of the present invention is a method for reducing the dose of chemotherapy treatment of a tumour, comprising applying an implant in the vicinity of said tumour prior to chemotherapy.

Where radiotherapy and chemotherapy are administered, the implant of the present invention may be used to reduce both radiotherapy and chemotherapy doses. A typical chemotherapy and/or radiotherapy dose may be about 20%, 30%, 40%, 50%, 60%, 70%, 80%, 90% less than the dose normally applied to a tumour, in view of the size, location and other factors. It may be a value in the range between any two of the aforementioned values. Preferably, the dose is between 20 and 70% less than the normal dose.

Another embodiment of the present invention is a method for sensitising proliferating cells (e.g. tumour) to radiotherapy, comprising applying an implant in the vicinity of said tumour prior to radiotherapy.

Another embodiment of the present invention is a method for sensitising proliferating cells (e.g. tumour) to chemotherapy, comprising applying an implant in the vicinity of said tumour prior to chemotherapy.

According to one aspect of the invention, the implant is applied to a subject at least 8 hours, 10 hours, 12 hours, 14 hours, 16 hours, 18 hours, 20 hours, 22 hours, 1 day, 2 days, 3 days, 4 days, 6, days, 8 days, 10 days, 12 days, 14 days, 3 weeks or 4 weeks before the start of radio- and/or chemotherapy, or for a period between any two of the aforementioned periods. Preferably, the implant is in place for between 12 hours to 4 weeks before commencement of radio- and/or chemotherapy.

### EXAMPLES

The invention is illustrated by the following non-limiting examples. They illustrate the effectiveness of a selection of glycolysis, TCA cycle, oxidative phosphorylation inhibitors described above. The inhibitory properties of the other inhibitors not mentioned in the examples are known, and the skilled person may readily substitute the exemplified inhibitors with equivalent pathway inhibitors such as listed above

A 35 y old woman presents with a 1 cm diameter tumour located in the upper external quadrant of her right breast. MRI examination does not show enlarged nodes in the corresponding axillar area. A PET-CT examination confirms the unicity of the breast tumour location with a SUV (Standard Uptake Value) of 7 for ¹⁸-FDG. The patient undergoes a tumorectomy and a sentinel lymph node resection. The histological analysis does not show the presence of tumour cells in the sentinel lymph node and confirms a grade 1 ductal carcinoma, without lymphovascular space invasion. A cylindric 3.5 cm long implant, 2 cm diameter, with lateral holes allowing suturing the implant to the cavity walls, is set in place and sutured to the walls exactly at the level where the tumour was located. The implant is made from PEG/PLGA polymer, and contains 200 mg of FDG, 30 mg dinitrophenol and 3 mg of fluoroacetate all to be released in 4 weeks during the degradation period of the implant. The implant is recovered by some breast tissue and breast fat, creating a 2 cm space of breast tissue between the implant outer surface and the skin. The patient is referred to external radiotherapy for the treatment of the whole breast 3 weeks after the surgical excision. She receives 50 Gy with a 6 MeV accelerator, by 2 tangential fields. Normally, such a patient should also receive an external boost (overdosage) on the resection cavity area, in order to further decrease the recurrence rate at the level of the resection cavity. Indeed, it is shown that 90% of recurrences occur in the close vicinity of the tumour (in fact mainly within 1 cm from the resection cavity surface). The implant releases the active products during 4 weeks, during its degradation period and prepares potential isolated tumour cells, remaining in the cavity wall resection walls, to become more sensitive to external radiotherapy.

Literature data show that for patients less than 50 y old, undergoing breast conserving surgery and benefiting from external radiotherapy at a standard dose of 50 Gy, there a high risk to present a recurrence located mainly in the resection cavity area. Therefore, all such patients receive today an additional boost to the resection cavity area with a dose of 16 Gy in 8 sessions (8 days of treatment plus 2 days for the WE), which represents 10 days of additional treatment time. The use of the implant, which renders present isolated tumour cells more sensitive to radiotherapy, allows reducing the therapeutic cumulated dose from 66 Gy to 50 Gy, improving treatment quality and precision as well as the quality of life of the patient. The treatment time is shorter by nearly 2 weeks (5 vs 7 weeks). The patient is accurately followed up for recurrence.

A 65 y old patient is diagnosed with a prostate tumour, with a PSA value of 10.5 ng/ml. MR examination with an intra-rectal antenna shows a unique tumour node, 2 cm in diameter, located in the right lobe of the prostate, very close to the capsula, in the prostate apex. A PET CT examination is performed which shows that the lesion takes 11-Cacetate very actively. The surgeon proposes to the patient a robot guided nerve sparing prostatectomy (accurate surgical resection), which is slightly dangerous because of the risk of leaving tumour tissue in the resection cavity walls. Indeed, it is known that the lesion is located very close to the prostate capsule, which increases the risk of having invaded resection margins after the intervention. In order to reduce this risk the patient agrees to receive 1 ovoid hollow biodegradable implant, 3 cm long and 2 cm in diameter, which is sutured to the right side of the prostate resection cavity, corresponding to the area where the right lobe was located and where the tumour node was close to the capsule. Indeed, the histological examination reports the vicinity of the resection margins which may be considered as invaded by the tumour, in the former right lobe.

The implant degrades in 4 weeks, releasing simultaneously 4 mg of fluoroacetate or 10 mg of halo-pyruvate, 250 mg of 2-FDG, and 15 mg of rhodamine 123. The follow-up during 1 y does not show any increase in PSA values. The patient does not report any side effect induced by the implant.

A 45 y old patient, a heavy smoker and drunker, presents with a 5 cm long epidermoid carcinoma in the medium third of the oesophagus and a weight loss of 12 kgs in 6 months. The Ctscan and an echoendoscopy shows a 6 mm thick lesion invading oesophageal serosa. The patient complains of odynophagia since several months, evaluated at a level of 7 on a maximal pain scale of 10. A PET CT examination shows a high uptake of 18FDG strictly limited at the level of the tumour.

In order to render tumour cells more sensitive, 5 days before starting radiochemotherapy, a catheter in the shape of a nasopharyngeal feeding tube, on which a longitudinal balloon is fixed, is introduced in the oesophagus. This balloon is 8 cm long and 0.7 cm in diameter when inflated. The balloon is coated with a hydrogel layer, 2 mm thick, containing 300 mg of 2-FDG, and 30 mg of dinitrophenol. The balloon is positioned and inflated in regard of the tumour. The balloon walls remain in thigh contact with the tumour surface and 2-FDG and dinitrophenol diffuse into the tumour while the balloon is inflated. The relatively small diameter of the inflated balloon does not put the oesophageal wall under high tension which would induce pain. The balloon is kept inflated during 5 days before the start of the radiotherapy and chemotherapy regimens. The balloon can be deflated when patient feels discomfort and a balloon may be reintroduced in the oesophagus during the chemotherapy sessions, in order to further potentiate the therapeutic association. The patient is given a standard chemotherapy regimen for oesophageal cancer: taxotere 20 mg/m²/week one day a week for 4 weeks together with 5-FU, 300 mg/m2/24 h continuous infusion during 5 days during week 1 and 4. Radiation therapy is delivered during 4 weeks, giving 40 Gy in 2 Gy fractions. The patient is operated. The histological analysis does not reveal the presence of remaining tumour tissue. The inhibition of oxphos and glycolysis has rendered the tumour cells particularly sensitive to the radiochemotherapy. Moreover, the active substances are drained into the regional lymph nodes, rendering the tumour cells present inside these structures more sensitive as well. High sterilization rates may be achieved, higher than sterilization rates known until now. Indeed, the most potent combinations of preoperative radio-chemotherapy regimens achieve maximal sterilization rates in the range of 50%.

A 88 y old lady complains of a vaginal bleeding for 5 months. The MRI shows a superficial endometrial tumour located on the anterior uterine wall measuring 4 cm in diameter with a thickness of 8 mm. No pelvic lymph nodes are seen on the MRI. A gynaecological examination confirms the presence of a uterine bleeding and the endometrial biopsy confirms the presence of an endometroid carcinoma. The patient refuses surgery. The cervical canal is dilated under loco-regional anaesthesia and a deflated conical balloon, coated with a 3 mm thick hydrogel foil, is introduced inside the uterine cavity. The hydrogel foil is either slightly flexible, or it can be longer than the deflated balloon, in order to allow adequate extension of the foil, once the balloon is inflated. The hydrogel foil contains 500 mg of oxamate, 30 mg of dinitrophenol and 5 mg of fluoroacetate, foreseen to elute during 1 week. The inflated balloon is a cone which measures 4.5 cm of length and 4 cm in diameter at the cranial larger side of the balloon. The balloon catheter has a special valve system allowing keeping it hermetically closed. One week later, the catheter is deflated, the balloon is removed, the bleeding has stopped, and the patient is kept under observation. At six months the MRI shows a flattening of the endometrial structure. Bleeding has not recurred. It is foreseen to perform the same therapy would the bleeding recur.

1-58. (canceled)

59. A medical implant suitable for insertion into the cavity of a subject provided with a composition comprising at least one type of inhibitor of the TCA cycle and/or oxidative phosphorylation, and a slow release agent.

60. A method for inhibiting cell proliferation in a cavity of a subject comprising inserting a medical implant comprising a composition comprising at least one type of inhibitor of the TCA cycle and/or oxidative phosphorylation, and a slow release agent, into the cavity of the subject.

61. The method according to claim 60, further comprising treating the proliferating cells by radiotherapy, wherein said medical implant is administered into the proliferating cell mass prior to the radiotherapy.

62. The method according to claim 60, further comprising treating the proliferating cells by chemotherapy, wherein said medical implant is administered into the proliferating cell mass prior to the chemotherapy.

63. A kit comprising a) at least one medical implant suitable for insertion into the cavity of a subject and b) a composition comprising at least one type of inhibitor of the TCA cycle and/or oxidative phosphorylation, and a slow release agent.

64. The medical implant according to claim 59, wherein said implant comprises a material which is bioabsorbable in situ.

65. The medical implant according to claim 59, wherein said implant comprises a material which is non-bioabsorbable.

66. The medical implant according to claim 59, wherein the implant comprises an inflatable medical balloon.

67. The medical implant according to claim 66, wherein said balloon is at least partly coated with said composition.

68. The medical implant according to claim 66, wherein said balloon is at least partly covered with an expandable foil over an expandable region of the balloon, said foil provided with said composition.

69. The medical implant according to claim 68, wherein said foil is at least partly coated with said composition.

70. The medical implant according to claim 68, wherein said foil is formed from any of aliphatic polyester copolymers, elastomeric copolymers of epsilon-caprolactone and glycolide, elastomeric copolymers of E-caprolactone and lactide, copolymers of E-caprolactone and lactic acid, elastomeric copolymers of p-dioxanone and lactide, elastomeric copolymers of p-dioxanone and lactic acid, elastomeric copolymers of epsilon-caprolactone and p-dioxanone, elastomeric copolymers of p-dioxanone and trimethylene carbonate, elastomeric copolymers of trimethylene carbonate and glycolide, elastomeric copolymer of trimethylene carbonate and lactide, or elastomeric copolymer of trimethylene carbonate and lactic acid.

71. The medical implant according to claim 68, wherein said foil comprises a material which is bioabsorbable in situ.

72. The medical implant according to claim 68, wherein said foil is made from a material which is non-bioabsorbable.

73. The medical implant according to claim 66, wherein said balloon further comprises a catheter.

74. The medical implant according to claim 66, wherein said balloon further comprises one or more catheters positioned in a geometrical configuration inside the balloon in order to offer optimal dosimetry for a therapy using ionizing or non-ionizing radiation.

75. The medical implant according to claim 59, wherein said implant is a static implant.

76. The medical implant according to claim 75, wherein said static implant is at least partly coated with said composition.

77. The medical implant according to claim 59, wherein said slow release agent is any of magnesium alloys, poly(glycolic) acid, poly(lactic acid) or in general glycolic- and lactic acid based polymers, copolymers, polycaprolactones and in general, polyhydroxyl alkanoates, poly(hydroxy alcanoic acids), Poly(ethylene glycol), polyvinyl alcohol, poly(orthoesters), poly (anhydrides), poly(carbonates), polyamides, polyimides, polyimines, poly(imino carbonates), poly(ethylene imines), polydioxanes, polyoxyethylene (polyethylene oxide), poly (phosphazenes), polysulphones, lipids, polyacrylic acids, polymethylmethacrylate, polyacrylamides, polyacrylonitriles (Polycyanoacrylates), polyHEMA, polyurethanes, polyolefins, polystyrene, polyterephthalates, polyethylenes, polypropylenes, polyether ketones, polyvinylchlorides, polyfluorides, silicones, polysilicates (bioactive glass), siloxanes (Poly dimethyl siloxanes), hydroxyapatites, lactide-capronolactone, natural and non natural polyamino acids, poly β-aminoesters, albumins, alginates, cellulose/cellulose acetates, chitin/chitosan, collagen, fibrin/fibrinogen, gelatin, lignin, protein based polymers, Poly(lysine), poly (glutamate), poly(malonates), poly(hyaluronic acids), Polynucleic acids, polysaccharides, poly (hydroxyalkanoates), polyisoprenoids, starch based polymers, copolymers thereof, linear, branched, hyperbranched, dendrimers, crosslinked, functionalized derivatives thereof, hydrogels based on activated polyethyleneglycols combined with alkaline hydrolyzed animal or vegetal proteins.

78. The medical implant according to claim 59, wherein at least one type of inhibitor of the TCA cycle and/or oxidative phosphorylation is encapsulated in a micro-capsule or nano-capsule.

79. The medical implant according to claim 78, wherein the nano-capsule comprises any of a copolymer poly(ethylene oxide) with poly(L-Lactic acid) or with poly(beta-benzyl-L-aspartate); copolymer with poly(lactide-co-glycolide)-[(propylene oxide)-poly(ethylene oxide)]; polyphosphazene derivatives; a poly(ethylene glycol) coated nanosphere; a poly(isobutylcyanoacrylate) nanocapsules poly(gamma-benzyl-L-glutamate)/(poly(ethylene oxide); chitosan-poly(ethylene oxide) nanoparticles; o-carboxymethylate chitosan, or a solid lipid nanosphere.

80. The medical implant according to claim 78, wherein the micro-capsule comprises any of chitosan; a coated alginate microsphere; an N-(aminoalkyl) chitosan microsphere; a chitosan/calcium alginate bead, a poly(adipic anhydride) microsphere; a gellan-gum bead; a poly(D, L-lactide-co-glycolide) microsphere; an alginate-poly-L-lysine microcapsule; a crosslinked chitosan microsphere; a chitosan/gelatin microsphere; a crosslinked chitosan network bead with spacer groups; an aliphatic polyester; a 1,5-diozepan-2-one microsphere; a D,L-dilactide microsphere; a triglyceride liposphere; a polyelectrolyte complex of sodium alginate chitosan; a polypeptide microcapsule, or an albumin microsphere.

81. The medical implant according to claim 59, wherein at least one of said inhibitors is coupled to a solubilizing agent.

82. The medical implant according to claim 81, wherein said solubilizing agent is cholesterol or a derivative thereof.

83. The medical implant according to claim 82, wherein said cholesterol derivative is any of cholesteryl-halogenated acetate.

84. The medical implant according to claim 81, wherein said solubilizing agent is vitamin A or a derivative thereof.

85. The medical implant according to claim 84, wherein the derivative of vitamin A is formula (IV) or (V): 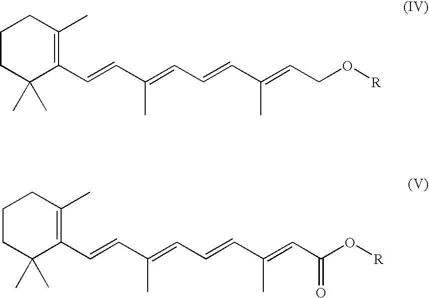
wherein R is selected from the group consisting of halogenated acetate.

86. The medical implant according to claim 59, wherein said inhibitor of the TCA cycle inhibits at least one enzyme from the group consisting of pyruvate dehydrogenase, citrate synthase, aconitase, isocitrate lyase, alpha-ketoglutarate dehydrogenase complex, succinyl CoA synthetase, succinate dehydrogenase, fumarase, malate synthase, glutaminase, glutamate dehydrogenase, pyruvate dehydrogenase complex and malate dehydrogenase.

87. The medical implant according to claim 86, wherein said inhibitor of the TCA cycle is any of halo-pyruvate, 3-fluoropyruvate, 3-chloropyruvate, 3-bromopyruvate, 3-iodopyruvate, fluoroacetate, fluorocitrate, halogenated citrate, bromocitrate, chlorocitrate, and iodocitrate,

88. The medical implant according to claim 59, wherein said inhibitor of oxidative phosphorylation is an inhibitor of enzyme complex V.

89. The medical implant according to claim 88, wherein said inhibitor of enzyme complex V is any of dinitrophenol, dinitrocresol, rhodamine, rhodamine 123, rhodamine 6G, a stereoisomer, tautomer, racemate, prodrug, metabolite thereof, or a pharmaceutically acceptable salt, base, ester or solvate thereof.

90. The medical implant according to claim 59, wherein said composition further comprises one or more polymers to facilitate attachment of the composition to the implant and/or facilitate slow release of the composition.

91. The medical implant according to claim 90, wherein said polymer is one or more of:
aliphatic polyesters, poly(amino acids), copoly(ether-esters), polyalkylenes oxalates, polyamides, poly(iminocarbonates), polyanhydrides, polyorthoesters, polyoxaesters, polyamidoesters, polylactic acid, polyethylene oxide, polycaprolactone, polyhydroxybutyrate valerates, polyoxaesters containing amido groups, poly(anhydrides), polyphosphazenes, silicones, biomolecules and blends thereof,
lactic acid D-,L- and meso lactide, epsilon-caprolactone, glycolide glycolic acid, hydroxybutyrate, hydroxyvalerate, para-dioxanone, trimethylene carbonate and its alkyl derivatives, 1,4-dioxepan-2-one, 1,5-dioxepan-2-one, 6,6-dimethyl-1,4-dioxan-2-one and polymer blends thereof,
polyanhydrides from diacids of the form HOOC—C₆H₄—O—(CH₂)m-O—C₆H₄—COOH wherein m is an integer in the range of from 2 to 8 and copolymers thereof with aliphatic alpha-omega diacids of up to 12 carbons,
naturally enzymatically or are hydrolytically unstable polymers, fibrin, fibrinogen, collagen, gelatin, glycosaminoglycans, elastin, and absorbable biocompatible polysaccharides such as chitosan, starch, fatty acids and esters thereof, glucoso-glycans and hyaluronic acid,
hydrogels
polyurethanes, silicones, poly(meth)acrylates, polyesters, polyalkyl oxides, polyethylene oxide, polyvinyl alcohols, polyethylene glycols and polyvinyl pyrrolidone, as well as, hydrogels, hydrogels from crosslinked polyvinyl pyrrolidinone and polyesters, polyolefins, polyisobutylene and ethylene-alphaolefin copolymers, acrylic polymers, methacrylate and copolymers, vinyl halide polymers and copolymers, polyvinyl chloride, polyvinyl ethers, polyvinyl methyl ether, polyvinylidene, polyvinylidene fluoride, polyvinylidene chloride, polyacrylonitrile, polyvinyl ketones, polyvinyl aromatics, polystyrene, polyvinyl esters, polyvinyl acetate, copolymers of vinyl monomers with each other and olefins, ethylene-methyl methacrylate copolymers, acrylonitrile-styrene copolymers, ABS resins and ethylene-vinyl acetate copolymers, polyamides, Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose acetate butyrate, cellophane, cellulose nitrate, cellulose propionate, cellulose ethers, carboxymethyl cellulose and hydroxyalkyl celluloses, and combinations thereof,
polyamides of the form-NH—(CH₂)n-—CO— and NH—(CH₂)x-NH—CO—(CH₂)y-CO, wherein n is an integer in the range of from 6 to 13; x is an integer in the range of from 6 to 12; and y is an integer in the range of from 4 to 16,
bioabsorbable elastomers, aliphatic polyester elastomers,
elastomeric copolymers of epsilon-caprolactone and glycolide having a mole ratio of epsilon-caprolactone to glycolide of from about 35:65 to about 65:35,
elastomeric copolymers of epsilon-caprolactone and lactide, L-lactide, D-lactide blends thereof or lactic acid copolymers having a mole ratio of epsilon-caprolactone to lactide of from about 35:65 to about 90:10 or from about 90:10 to about 80:20,
elastomeric copolymers of p-dioxanone and lactide including L-lactide, D-lactide and lactic acid having a mole ratio of p-dioxanone to lactide of from about 40:60 to about 60:40,
elastomeric copolymers of epsilon-caprolactone and p-dioxanone having a mole ratio of epsilon-caprolactone to p-dioxanone of from about 30:70 to about 70:30,
elastomeric copolymers of p-dioxanone and trimethylene carbonate having a mole ratio of p-dioxanone to trimethylene carbonate of from about 30:70 to about 70:30,
elastomeric copolymers of trimethylene carbonate and glycolide having a mole ratio of trimethylene carbonate to glycolide of from about 30:70 to about 70:30,
elastomeric copolymer of trimethylene carbonate and lactide including L-lactide, D-lactide, blends thereof or lactic acid copolymers having a mole ratio of trimethylene carbonate to lactide of from about 30:70 to about 70:30 and blends thereof.

92. The medical implant according to claim 59, wherein:
the oxidative phosphorylation inhibitor is present in a quantity to primarily inhibit the oxidative phosphorylation pathway, and
the TCA cycle inhibitor, is present in a quantity to secondarily inhibit the TCA cycle.

93. The medical implant according to claim 59, wherein:
the TCA cycle inhibitor is present in a quantity to primarily inhibit the TCA cycle pathway, and
the oxidative phosphorylation inhibitor, is present in a quantity to secondarily inhibit the oxidative phosphorylation pathway.

94. A method of preparing a medical device for inhibiting cell proliferation comprising coating a composition comprising at least one type of inhibitor of the TCA cycle and/or oxidative phosphorylation, and a slow release agent onto a medical implant.

95. The method according to claim 60, wherein said cell proliferation is cancer.

96. The method according to claim 60, wherein said cell proliferation is restenosis or stenosis.

97. The method according to claim 60, wherein the implant is placed in a natural cavity.

98. The method according to claim 97, wherein said natural cavity is any of artery, vein, bronchial duct, biliary duct, esophagus, urethral duct, urethral duct, aeric tract, urogenital tract, nasopharyngeal area, pharynx, small and large bowels, rectum, the trachea, uterine cavity, uterine cervix, vagina, urethra or bladder.

99. The method according to claim 60, wherein the implant is placed in a resection cavity.

100. The method according to claim 99, wherein said cavity is selected from any of brain tumor resection, breast tumor resection, prostate cancer resection, muscle resection after a sarcoma, uterine laparoscopic myoma resection, head and neck resection cavities, tongue tumor resection, partial upper maxillary resection, liver tumor resection, kidney tumor resection, or bone tumor resection, scar cavity of a melanoma resection or scar of a cheloid resection, or any type of scar.

101. A method of treating cellular proliferation in a cavity of a subject comprising inserting an implant as defined in claim 59 into said cavity, said implant configured to contact at least part of the walls of the cavity.
